(19) United States
(12) Patent Application Publication
(10) Pub. No.: US 2005/0176009 A1
(43) Pub. Date: Aug. 11, 2005

(54) Polymorphic olfactory receptor genes and arrays, kits and methods utilizing information derived therefrom for genetic typing of individuals

(76) Inventors: Doron Lancet, Tel-Aviv (IL); Idan Menashe, Ramat Gan (IL); Orna Man, Kfar Givton (IL); Yoav Gilad, Rechovot (IL)

Correspondence Address: Martin Moynihan;Anthony Castorina, Suite 207, 2001 Jefferson Davis Highway, Arlington, VA, 22202, US

(21) Appl. No.: 10/511,278

(22) PCT Filed: Apr. 24, 2003
(86) PCT No.: PCT/IL03/00336

Related U.S. Application Data
(60) Provisional application No. 60/374,508, filed on Apr. 23, 2002.

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

An oligonucleotide comprising a nucleic acid sequence selected suitable for identifying an olfactory receptor gene or an allelic variant thereof is provided.

### FIELD AND BACKGROUND OF THE INVENTION

The present invention relates to polymorphic olfactory receptor genes and more particularly, to arrays, kits and methods utilizing information derived from these polymorphic sequences for genetic typing of individuals.

Olfactory transduction begins with the binding of an odorant ligand to a protein receptor on the olfactory neuron cell surface, thus initiating a cascade of reactions which results in the production of a second messenger and eventual depolarization of the cell membrane. This relatively straightforward and common signalling pathway is complicated by the fact that there are several thousand odorants, mostly low molecular weight organic molecules, and nearly one thousand different receptors.

ORs are members of the superfamily of membrane receptors characterized structurally by possessing seven transmembrane spanning helices, and functionally by being coupled to GTP-binding proteins. Although OR genes make up the largest sub-family of G-protein coupled receptors (GPCRs) most vertebrate odorant receptors are classified as “orphan” receptors having no identified ligand.

It is now clear that the members of this gene family have common ancestral origins which have undergone considerable divergence throughout evolution. Strong selective pressures have caused expansion and diversification of the OR gene repertoire, thereby modifying and honing the sense of smell in mammmals.

The diversified OR repertoire of mammals is needed in order to allow individuals to detect and discriminate between thousands of different odorant molecules. One of the most surprising features of human olfaction is that >60% of the OR genes bear one or more sequence disruptions, likely resulting in the inactivation of the encoded protein. There are indications that such massive OR pseudogenization is a relatively recent genomic process, likely to still be ongoing. Specific loss of OR genes resulted in an ensemble of only ˜400 functional ORs in humnas, likely leading to a decay in some aspects of olfactory faculties.

It is well known that human olfactory thresholds exhibits a very high degree of inter-individual variability. At one extreme is specific anosmia, an individual's incapacity to perceive an odorant detectable by many others. Less pronounced cases are termed specific hyposmia, which are characterized by an increased threshold (diminished sensitivity) to a particular odorant in certain members of the population. At the other end of the spectrum is specific hyperosmia, enahnced sensitivity to a specific odorant.

These phenomena can also be described in terms of quantitative traits, whereby the human population displays a threshold distribution in which specific anosmia and hyperosmia form the two extreme ends of the distribution. Because many dozens of odorants manifest such variability, it turns out that practically every human being is specifically anosmic or hyperosmic to one or more odorants.

Olfactory receptors interact with a diverse array of volatile molecules. It is widely accepted that every odorous molecule binds to several ORs and vise versa. This binding pattern generates a unique combinatorial code that generates a specific aroma for each odorant and enables the organism to distinguish it from other molecules. This system is highly sensitive and allows to discriminate between two protein isomers and at times even between two optical enantiomers.

It was suggested that odorant binding patterns correspond to a particular receptor affinity-binding distribution (RAD) [Lancet et al. ( 1993) PNAS (30) 3715-3719]. According to this model, the probability that an odorant will bind to its receptor(s) can be described through a distribution of binding affinities. Most of these binding affinities are weak, and only few have biological significance of which, the strongest affinity receptor determines the odorant threshold sensitivity. Thus, if such a receptor is missing from a receptor repertoire of an individual, the threshold would be defined by the next strongest affinity, to an existing receptor. In the case of a large receptor ensemble, a typical scenario would be that an odorant would be bound by several ORs with closely spaced affinity values. Consequently, it could lead to such a high level of functional redundancy, that a loss of an odorant highest-affinity receptor is unlikely to generate a recognizable olfactory deficit. This appears to be the case in macrosmatic organisms, (e.g. the mouse) where relatively few cases of clearly discernible threshold variations have been reported.

In sharp contrast, in humans, where the OR repertoire has diminished significantly in size, affinity values would tend to be more widely spaced, and threshold variations would become prevalent. In these cases, an excessive decrease of specific odorant detection is expected in individuals who have lost the receptor exhibiting the strongest binding to a particular odorant.

Such a hypothesis suggests that specific sensitivity to odorants present at below threshold concentrations may be treated as a single gene trait. In addition to such quantitative effects, it is worthwhile to consider the effect of a single gene inactivation on our qualitative olfactory cues. If a unique aroma of a given odorant is a product of signals from various ORs, a single change in this OR spectrum could modify the way the odorant signal is interpreted in the brain.

While reducing the present invention to practice, the present inventors have uncovered polymorphic OR genes which exhibit loss or reduced function in receptor capacity. The present inventors have also demonstrated that the occurrence of these allelic variations differ in individuals from different ethnic backgrounds thereby suggesting that polymorphism in OR genes contributes to differences in smell perception of individuals.

### SUMMARY OF THE INVENTION

According to one aspect of the present invention there is provided an oligonucleotide comprising a nucleic acid sequence selected suitable for identifying an olfactory receptor gene or an allelic variant thereof, the olfactory receptor gene being selected from the group consisting of SEQ ID NOs: 79-104.

According to further features in preferred embodiments of the invention described below, the oligonucleotide further comprising a detectable moiety attached to the nucleic acid sequence.

According to still further features in the described preferred embodiments the detectable moiety is selected from the group consisting of a dye, a fluorophore, an enzyme, a ligand and a radioisotope.

According to still further features in the described preferred embodiments the oligonucleotide is selected from the group consisting of SEQ ID NOs: 1-78.

According to still further features in the described preferred embodiments the oligonucleotide is an SNP-specific oligonucleotide.

According to still further features in the described preferred embodiments the oligonucleotide is a primer extension oligonucleotide.

According to another aspect of the present invention there is provided a kit for identifying an olfactory receptor gene and/or an allelic variant thereof, the kit comprising at least oligonucleotide having a nucleic acid sequence selected suitable for identifying the olfactory receptor gene and/or the allelic variant thereof.

According to still further features in the described preferred embodiments the kit further comprising reagents suitable for detecting identification of the olfactory receptor gene and/or the allelic variant thereof by the at least one oligonucleotide.

According to still further features in the described preferred embodiments the kit further comprising packaging material identifying the at least oligonucleotide as being utilizable in detecting the olfactory receptor gene and/or the allelic variant thereof.

According to still further features in the described preferred embodiments the at least one oligonucleotide is selected from the group consisting of SEQ ID NOs: 1-78.

According to still further features in the described preferred embodiments the at least oligonucleotide includes a detectable moiety attached to the nucleic acid sequence.

According to still further features in the described preferred embodiments the detectable moiety is selected from the group consisting of a dye, a fluorophore, an enzyme, a ligand and a radioisotope.

According to yet another aspect of the present invention there is provided an array for detecting the presence or absence of at least one allelic variant of an olfactory receptor gene in a subject, the array comprising at least one oligonucleotide being contained in or attached to a support, the at least oligonucleotide having a nucleic acid sequence selected suitable for specifically identifying the at least one allelic variant of the olfactory receptor gene.

According to still further features in the described preferred embodiments the array further comprising at least one additional oligonucleotide having a nucleic acid sequence selected suitable for specifically identifying the olfactory receptor gene.

According to still further features in the described preferred embodiments the olfactory receptor gene is selected from the group consisting of SEQ ID NOs: 79-104.

According to still further features in the described preferred embodiments the at least one oligonucleotide is selected from the group consisting of SEQ ID NOs: 27-78.

According to still another aspect of the present invention there is provided an array for typing a subject according to presence or absence of allelic variants of olfactory receptor genes, the array comprising a plurality of oligonucleotides each being attached to a support, the plurality of oligonucleotides include at least one typing oligonucleotide having a sequence selected suitable for specifically identifying presence or absence of a specific allelic variant of a specific olfactory receptor gene in the subject.

According to still further features in the described preferred embodiments the plurality of oligonucleotides also include at least one reference oligonucleotide having a sequence selected suitable for specifically identifying the specific olfactory receptor gene.

According to still further features in the described preferred embodiments the support is a chip.

According to still further features in the described preferred embodiments the at least one typing oligonucleotide is selected from the group consisting of SEQ ID NOs: 27-78.

According to still further features in the described preferred embodiments wherein the at least one reference oligonucleotide is selected from the group consisting of SEQ ID NOs: 1-26.

According to an additional aspect of the present invention there is provided a method of typing a subject according to presence or absence of allelic variants of olfactory receptor genes, the method comprising detecting the presence or absence of at least one allelic variant of an olfactory receptor gene in a biological sample of the subject thereby typing the subject.

According to still further features in the described preferred embodiments further comprising detecting the presence or absence of the olfactory receptor gene in :the biological sample of the subject.

According to still further features in the described preferred embodiments the olfactory receptor gene is selected from the group consisting of SEQ ID NOs: 79-104.

According to still further features in the described preferred embodiments the detecting the presence or absence of at least one allelic variant of the olfactory receptor gene is effected using at least one oligonucleotide selected from the group consisting of SEQ ID NOs: 27-78.

According to still further features in the described preferred embodiments the detecting the presence or absence of the olfactory receptor gene is effected using at least one oligonucleotide selected from the group consisting of SEQ ID NOs: 1-26.

According to still further features in the described preferred embodiments the detecting the presence or absence of at least one allelic variant of the olfactory receptor gene is effected by detecting DNA and/or mRNA sequences.

According to still further features in the described preferred embodiments the detecting the presence or absence of at least one allelic variant is effected using at least one oligonucleotide selected from the group consisting of SEQ ID NOs: 27-78.

According to still further features in the described preferred embodiments the detecting the presence or absence of the olfactory receptor gene is effected using at least one oligonucleotide selected from the group consisting of SEQ ID NOs: 1-26.

According to yet an additional aspect of the present invention there is provided a nucleic acid construct comprising a polynucleotide encoding an olfactory receptor gene and/or an allelic variant thereof, the olfactory receptor gene being selected from the group consisting of SEQ ID Nos: 79-104 and a promoter for directing transcription of the olfactory receptor gene or the allelic variant thereof in a cell.

According to still an additional aspect of the present invention there is provided a cell comprising the nucleic acid construct.

According to still further features in the described preferred embodiments the cell is a mammalian cell.

The present invention successfully addresses the shortcomings of the presently known configurations by providing polymorphic olfactory receptor genes and arrays, kits and methods utilizing information derived from these polymorphic sequences for genetic typing of individuals.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates the olfactory receptor (OR) gene cluster on human chromosome 17p13.3. Genes are positioned in their correct order and orientation, with exons marked as triangles and introns marked as squares. The regions that were sequenced are filled. The serial numbers of individual SNPs analyzed within each OR gene are shown inside ovals and are corresponding to the list detailed elsewhere (http://bioinfo.weizmann.ac.i1/˜menashe/OR17_SNPs.html). The span of the three intervals used for Clark's algorithm is indicated with Roman numerals I-III.

FIG. 2 illustrates the haplotypes of 30 diploid individuals, as marked in the left column. The 40 polymorphic sites are indicated by their position in the different genes on the top row. The rare variant is represented by a dark square. The intact and disrupted alleles of the putatively functional SNPs are marked by green and red squares, respectively. The segregating pseudogene haplotypes are shown in order of their increasing disruption level from top to bottom. The normalized fraction of each haplotype in the Pygmies was as follows: I, 0.1; II. 0.0; Il. 0.6; IV. 1.0; V. 0.9; VI. 0.9; VII. 0.9.

FIG. 3 illustrates a linkage disequilibrium diagram across the OR cluster. SNP numbers are as in FIGS. 1 and 2. Dark blue squares Indicate pairs in significant LD indicated by both the Clark and EM algorithms. Other colors indicate pairs in significant LD indicated only by one algorithm: Clark's (light blue) or EM (yellow). The significance level in all pairs labeled in color was p<0.05 using Fisher's exact test. The position of the SNPs relative to the OR genes and the Clark's algorithm analysis intervals are indicated near the diagonal.

FIGS. 4a-e are scatter-plots of the decay of LD with physical distance. Each point is the D′-value for a pair of sites separated by a physical distance indicated on the abscissa. A linear-fit trend-line for the data is plotted. The significance of the data was calculated using a permutation test. The decay of LD for the entire sample is shown at a distance of 124 Kb (R=−0.53, P<0.01). Pairs with significant (P<0.05) D′-pairs are in red triangles (FIG. 4a).

FIGS. 4b-e illustrate the decay of LD for the specific ethnogeographic groups: The Pygmies (FIG. 4b), the Yemenite Jews (FIG. 4c), the Bedouins (FIG. 4d) and the Ashkenazi Jews (FIG. 4e).

FIGS. 5a-b illustrate the observed individual OR genotypes in African-Americans (FIG. 5a) and non-Africans (FIG. 5b). Red indicates homozygous OR disruption, dark green denotes homozygous intact OR and light green is for heterozygotes. Both individuals (rows) and OR loci (columns) are ordered according to their disruption level. Disruption type is indicated on top:. N—nonsense mutation, D—Deletion/Insertion and S—mis-sense substitution at a highly conserved site. The 178 putative phenotypes seen in the 189 individuals based on recessive inheritance are in accordance with a computed expectation value assuming Hardy-Weinberg equilibrium and based on the allele frequencies present.

FIG. 6 illustrates population differences in the frequency of intact OR alleles. The values for Caucasians are plotted as dark bars, and those for the Pygmies as empty bars. OR genes are indicated by their official Human Genome Organization human gene nomenclature symbols (Glusman et al. 2000).

FIG. 7 illustrates the phenotype distribution of the OR gene cluster for different ethnic groups. Shown are the population frequencies of the counts of deduced functional loci in African Americans (light baris) and non-Africans (dark bars). OR loci were considered intact if the individual carried at least one copy of the intact allele. The broken line curves are Gaussian fits with μ=20.06 and σ=1.73 for African Americans and μ=19.40 and σ=1.61 for non-Africans. The statistical significance of the difference in the number of intact ORs between African-American and non-African individuals was assessed by a Man-Whitney-U test, P=0.0022, or by a χ² test, P=0.014. A comparable P-value was obtained also when the functional allele was not assumed as dominant and the three different genotypes were not used at each locus (Man-Whitney-U test, P=0.0001, or χ², P=0.015).

### DESCRIPTION OF THE PREFERRED EMBODIMENTS

The present invention is of polymorphic olfactory genes and arrays, kits and methods utilizing information derived therefrom for typing the olfactory sensitivity of individuals or populations.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Evidence collected over the past decade suggests a clear genetic basis for human olfactory threshold variations. Characterization of human OR genes over the past decade has paved the way for creating a direct link between olfactory phenotypes and OR genotypes. However, to date, such linkage has not been established.

While reducing the present invention to practice, the present inventors have uncovered a set of novel OR genes (SEQ ID NOs: 79-104) in which a single nucleotide polymorphism (SNP, i.e., variation from the most frequently occurring base at a particular nucleic acid position) segregates between intact and disrupted alleles in the human population (see Example 2 of the Examples section).

These alternative forms of the genes, hereinafter also referred to as allelic variants, exhibit remarkable diversity among human population (see the Examples section hereinbelow) by which every human is characterized with a unique set of intact and disrupted ORs. More intriguingly, significant differences in intact ORs count were observed between different populations suggesting that the inter-individual olfactory differences might be more notable among groups of diverse ethnicities.

As further described in the Examples section which follows, the present inventors employed two screening approaches to find human polymorphic OR genes. In the first approach, OR pseudogenes that have only one open reading frame disruption were sought [Glusman (2001) Genome Res. 11:685-702]. Fifty of these sequences were sequenced in a chimpanzee since differential pseudogene state between the two higher apes would suggest recent evolutionary events which might generate human polymorphism. The second approach included querying Celera's human SNP database for genetic variations, which might affect protein integrity. 51 OR loci obtained from these screenings where genotyped in 189 individuals from several ethnic origins (see Example 1).

As illustrated in the Example section which follows, 26 OR loci were found to segragate between an intact and disrupted alleles in the study group, giving rise to an unprecedented number of unique genotypic patterns i.e., haplotypes.

Extrapolation has estimated the number of such segregating pseudogenes (SPGs) in the entire human genome at approximately 60 genes, which cover approximately 15% of the human functional OR repertoire. This number of SPGs in rough agreement with the reported count of different modes of human odorant-specific sensory deficits.

Thus, the presently discovered genotypic disparity is an important tool in the elucidation and possibly characterization of olfactory variation in humans.

Thus, according to one aspect of the present invention there is provided a method of typing a subject according to presence or absence of allelic variants of the olfactory receptor genes of the present invention.

The method is effected by detecting the presence or absence of at least one allelic variant of an olfactory receptor gene in a biological sample of the subject, thereby typing the subject.

As used herein the term “subject” refers to a mammalian subject which is preferably a human.

As used herein, the phrase “biological sample” refers to a sample of tissue or fluid isolated from a individual, including but not limited to, for example, plasma, serum, spinal fluid, lymph fluid, the external sections of the skin, respiratory, intestinal, and genitourinary tracts, tears, saliva, milk, semen and organs.

Preferably, the biological sample of this aspect of the present invention is obtained from the olfactory neuroephithlium, located at the upper area of each nasal chamber adjacent to the cribriform plate, superior nasal septum, and superior-lateral nasal wall. Methods of obtaining olfactory epithelium are well known in the art.

Detecting the presence or absence of at least one allelic variant of an olfactory receptor gene according to this aspect of the present invention can be effected at at the nucleic acid sequence level of the OR gene (i.e., DNA or transcribed RNA) or at the protein level, i.e. polynucleotides expressed from the OR gene.

Numerous hybridization based techniques are known in the art for detecting nucleic acid sequence variations. A given nucleic acid sequnece or any number of sequences can be detected by hybridization to a specific probe. Such probes may be cloned DNAs or fragments thereof, RNA, typically made by in-vitro transcription, or oligonucleotides; oligonucleotides can also be used as primers in amplification based detection approaches (i.e., PCR).

As used herein, the term “oligonucleotide” refers to a single stranded or double stranded oligomer or polymer of ribonucleic acid (RNA) or deoxyribonucleic acid (DNA) or mimetics thereof This term includes oligonucleotides composed of naturally-occurring bases, sugars and covalent internucleoside linkages (e.g., backbone) as well as oligonucleotides having non-naturally-occurring portions which function similarly. Such modified or substituted oligonucleotides are often preferred over native forms because of desirable properties such as, enhanced affinity for nucleic acid target and increased stability in the presence of nucleases.

The oligonucleotides of the present invention preferably include nucleic acid sequences that are substantially homologous to nucleic acid sequences that flank and/or extend across the SNPs of the present invention (see Table 5 of the Examples section).

Examples of oligonucleotides which can be used to identify both ORs of the present invention and allelic variants thereof are presented in Table 6 of the Examples section which follows (SEQ ID NOs. 1-78).

Oligonucleotides generated by the teachings of the present invention may be used in any modification of nucleic acid hybridization based techniques. As such the oligonucleotides of the present invention can correspond to any cDNA, mRNA and genomic sequences regions which stretch across 10 bp, 20 bp, 30 base pairs (bp), or even 40, 50, or 100 bp, or longer. Oligonucleotides of 10 to 1000 bp or even more, may have utility as hybridization probes in a variety of hybridization techniques including Southern and Northern blotting. The total size of oligonucleotide used, as well as the size of complementary sequences depend on the intended use or type of detection employed.

In general, the oligonucleotides of the present invention may be generated by any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and as such is not further described herein.

The oligonucleotides of the present invention may comprise heterocylic nucleosides consisting of purines and the pyrimidines bases, bonded in a 3′ to 5′ phosphodiester linkage.

Preferably oligonucleotides utilized by the present invention are those modified in either backbone, internucleoside linkages or bases, as is broadly described hereinunder. Such modifications can oftentimes facilitate oligonucleotide uptake and resistivity to intracellular conditions.

Examples of oligonucleotides which can be used with this aspect of the present invention include oligonucleotides containing modified backbones or non-natural internucleoside linkages. Oligonucleotides having modified backbones include those that retain a phosphorus atom in the backbone. U.S. Pat. Nos.: ,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466, 677; 5,476,925; 5,519,126; 5,536,821; 5,541,306; 5,550,111; 5,563,253; 5,571,799; 5,587,361; and 5,625,050 disclose oligonucleotide synthesis approaches which can be utilized by the present invention.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones include backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include,.but are not limited to, U.S. Pat. Nos. 5,539,082; 5,714,331; and 5,719,262, each of which is herein incorporated by reference. Other backbone modifications, which can be used in the present invention are disclosed in U.S. Pat. No: 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of a denine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, &-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine. and 8-azaadenine, 7-deazaguanine and 7-deazaadenine and 3-deazaguanine and 3-deazaadenine. Further bases include those disclosed in U.S. Pat. No: 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N -6 and O-6 substituted purines, including 2-aminopropyladenine, 5-opynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

It will be appreciated that it is not necessary for all positions in a given oligonucleotide molecule to be uniformly modified, and in fact more than one of the aforementioned modifications may be incorporated in a single compound or even at a single nucleoside within an oligonucleotide.

The oligonucleotides of the present invention are contacted with the biological sample to generate oligonucleotide-nucleic acid sequence specific hybrids. Contacting the oligonucleotides of the present invention with the biological sample is effected by stringent, moderate or mild hybridization (as used in any polynucleotide hybridization assay such as northern blot, dot blot, RNase protection assay, RT-PCR and the like). Wherein stringent hybridization is effected by a hybridization solution of 6×SSC and 1% SDS or 3 M TMACI, 0.01 M sodium phosphate (pH 6.8), 1 mM EDTA (pH 7.6), 0.5% SDS, 100 [g/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ; moderate hybridization is effected by a hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon spern DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; whereas mild hybridization is effected by a hybridization solution of 6×SSC and 1% SDS or 3 M TMACI, 0.01 M sodium phosphate (pH 6.8), 1 mM EDTA (pH 7.6), 0.5% SDS, 100 μg/ml denatured salnon sperm DNA and 0.1% nonfat dried milk, hybridization temperature of 37° C., final wash solution of 6×SSC and final wash at 22° C.

In general, quantifying hybridization complexes is well known in the art and may be achieved by any one of several approaches. These approaches are generally based on the detection of a label or marker, such as any radioactive, fluorescent, biological or enzymatic tags or labels of standard use in the art. A label can be applied on either the oligonucleotide probes or nucleic acids derived from the biological sample.

The following illustrates a number of labeling methods suitable for use in the present invention. For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, FluorX (Amersham) and others [ e.g., Kricka et al. (1992), Academic Press San Diego, Calif.] can be attached to the oligonucleotides. It will be appreciated that pairs of fluorophores are chosen when distinction between two emission spectra of two oligonucleotides is desired or optionally, a label other than a fluorescent label is used. For example, a radioactive label, or a pair of radioactive labels with distinct emission spectra, can be used [Zhao et al. (1995) Gene 156:207]. However, because of scattering of radioactive particles, and the consequent requirement for widely spaced binding sites, the use of fluorophores rather than radioisotopes is more preferred.

The intensity of signal produced in any of the detection methods described hereinabove may be analyzed manually or using hardware and software suited for such purposes.

Alternatively, detection of allelic variants according to this aspect of the present invention can be effected at the protein level provided that the allelic variation is expressed in the amino acid sequence of the OR. For example, OR1E3P (SEQ ID NO: 84) includes a single base deletion in nucleotide coordinate 54 which causes a frame shift resulting in a premature stop codon. Such variation can be detected at the protein level based on, for example, electrophoretic mobilization, N-terminal Edman sequencing or antibody recognition. OR3A1 (SEQ ID NO: 89) is another example wherein a single nucleotide substitution (G>A) results in an amino acid substitution (Arginine>Glutmaine) in a highly conserved region of the ORs [i.e., the DRY motif Reich (1998) Proc. Natl. Acad. Sci. USA 95:8119-23; Risch (1996) Science 273:1516-7; Rouquier(2000) Proc. Natl. Acad. Sci. USA 97:2870-4]. In this case too, such sequence variation can be detected using a specific antibody.

Polypeptide sequences can be extracted from the biological sample using a variety of methods which are well known to the ordinary skilled in the art. The protein can be isolated by conventional means of protein biochemistry and purification to obtain a substantially pure product, i.e., 80, 95 or 99% free of cell component contaminants, as described in Jacoby, Methods in Enzymology Volume 104, Academic Press, New York (1984); Scopes, Protein Purification, Principles and Practice, 2n Edition, Springer-Verlag New York (1987), and Deutscher (ed), Guide Protein Purification, Methods in Enzymology, Vol. 182 (1990).

As used herein the term “antibody”, refers to an intact antibody molecule and the phrase “antibody fragment” refers to a functional fragment thereof, such as Fab, F(ab′)₂, and Fv that are capable of binding to macrophages. These functional antibody fragments are defined as follows: (i) Fab, the fragment which contains a monovalent antigen-binding fragment of an antibody molecule, can be produced by digestion of whole antibody with the enzyme papain to yield an intact light chain and a portion of one heavy chain; (ii) Fab′, the fragment of an antibody molecule that can be obtained by treating whole antibody with pepsin, followed by reduction, to yield an intact light chain and a portion of the heavy chain; two Fab′ fragments are obtained per antibody molecule; (iii) (Fab′)₂, the fragment of the antibody that can be obtained by treating whole antibody with the enzyme pepsin without subsequent reduction; F(ab′)₂ is a dimer of two Fab′ fragments held together by two disulfide bonds; (iv) Fv, defined as a genetically engineered fragment containing the variable region of the light chain and the variable region of the heavy chain expressed as two chains; (v) Single chain antibody (“SCA”), a genetically engineered molecule containing the variable region of the light chain and the variable region of the heavy chain, linked by a suitable polypeptide linker as a genetically fused single chain molecule; and (vi) Peptides coding for a single complementarity-determining region (CDR).

Methods of generating antibodies (i.e., monoclonal and polyclonal) are well known in the art. Antibodies may be generated via any one of several methods known in the art, which methods can employ induction of in vivo production of antibody molecules, screening immunoglobulin libraries or panels of highly specific binding reagents as disclosed [Orlandi D. R. et al. (1989) Proc. Natl. Acad. Sci. 86:3833-3837, Winter G. et al. (1991) Nature 349:293-299] or generation of monoclonal antibody molecules by continuous cell lines in culture. These include but are not limited to, the hybridoma technique, the human B-cell hybridoma technique, and the Epstein-Bar-Virus (EBV)-hybridoma technique [Kohler G., et al. (1975) Nature 256:495-497, Kozbor D., et al. (1985) J. Immunol. Methods 81:31-42, Cote R. J. et al. (1983) Proc. Natl. Acad. Sci. 80:2026-2030, Cole S. P. et al. (1984) Mol. Cell. Biol. 62:109-120].

According to preferred embodiments of this aspect of the present invention, subject typing is effected using a plurality of oligonucleotides or antibodies which are attached to a solid substrate configured as a microarray. Microarrays are known in the art and consist of a surface to which probes that correspond in sequence to gene products (e.g., cDNAs, mRNAs, cRNAs, polypeptides, and fragments thereof), can be specifically hybridized or bound at a known position (i.e., regiospecificity).

Several methods for attaching the oligonucleotides to a microarray are known in the art including but not limited to glass-printing, described generally by Schena et al., 1995, Science 270:467-47, photolithographic techniques [Fodor et al. (1991) Science 251:767-773], inkjet printing, masking and the like.

Antibody arrays are also known in the art and disclosed in U.S. Pat. No. 6,329,209.

Since the allelic variants described herein may exhibit modified odorant specificity, it may be advantageous to type the odorant ligand of such ORs. Thus, the present invention also envisages nucleic acid constructs which include the polymorphic OR sequences of the present invention and may be used to express these sequences in a variety of host cells.

Cloning of the OR sequences of the present invention into nucleic acid expression constructs can be effected using commercially available eukaryotic expression vectors or derivatives thereof. Examples of suitable vectors include, but are not limited to pcDNA3, pcDNA3.1 (±), pGL3, PzeoSV2 (±), pSecTag2, pDisplay, pEF/myc/cyto, pCMV/myc/cyto, pDR3.1, pSinRepS, DH26S, DHBB, pNMT1, pNMT41, pNMT81, which are available from Invitrogen, pMbac, pPbac, pBK-RSV and pBK-CMV which are available from Stratagene, pTRES which is available from Clontech.

Any promoter and/or regulatory sequences included in the expression vectors described above can be utilized to direct the transcription of the OR genes of the present invention.

Preferably, the promoter that is selected according to the host cells or tissues of interest. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J; 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). Promoters for expression of the polynucleotide can also be developmentally-regulated promoters such as the murine homeobox promoters [Kessel et al. (1990) Science 249:374-379) or the fetoprotein promoter [Campes et al. (1989) Genes Dev. 3:537-546].

The nucleic acid construct can be introduced into the cell via any transformation method known in the art.

Once expressed, OR sequences of the present invention may be used to identify their congnate ligands. Methods of identifying OR ligands are disclosed in U.S. Pat. No. 5,993,778.

Since the above described probes and array enable typing of individuals according to the OR alleles carried or expressed thereby and since as is clearly shown in the Examples section which follows the prevalence of OR alleles varies from one population to another, the OR typing methodology described hereinabove can be utilized to determine an olfactory sensitivity and range of populations, subpopulations and even individuals.

Cross-cultural and sexual differences in olfactroy finctions have been demonstrated in the past. A number of researchers evaluated the olfactory sensitivity and preferences of natives from primitive tribes in the late nineteenth and early twentieth centuries.

Lombroso and Carrara [(1896/1897) Atti. Soc. Romana Antropol. 4:103] presented.a group o f Dinkas of the S udan with dilutions of clove oil ranging from 1:200˜1:50,000. These authors reported that recognition did not occur for concentrations lower than 1:2000 and that three Dinkas were unable to recognize the material even at the highest concentrations.

Myers [(1903) Brit. J. Psychol. 1:117] used aqueous solutions of camphor to evaluate the olfactory sensitivity of a group of Murray Islanders. In addition, these individuals were asked to describe the odors of perfum es and other scents and to elucidate whether they liked or disliked them. The average olfactory acuityof the islanders was reported as being slightly higher than that of Scottish control subjects and their likes and dislikes were noted as being quite similar.

Using a Zwaardemaker olfactometer, Grijns [(1906) Arch. Physiol. 509:517] compared sensitivity of a small group of Javanese subjects to those of some Europeans. He concluded that the Javanese were about twice as sensitive to the three test materials i.e., acetic acid, ammonia and phenol.

Contemporary studies devoid of inherent limitations of the above-described studies [i.e., the use of deadly poisons (i.e., phenol) and trigeminal stimulative materials (i.e., ammonia and acetic acid as test compounds], suggest that cross-cultural differences in odor preferences are goverened by still unknown genetically determined factors which interact with the well-known influences of experience on small perception to produce a variety of chemosensory experiences [Schleidt (1981) J. Chem. Ecol. 7:19; Davies and Pangbom (1985) Odor pleasantness judgments compared among samples from 20 nations using microfragrances. Seventh Annual Meeting of the association for Chemoreception Sciences, Sarasota, Fla., April 24-28].

As is clearly shown in the Examples section which follows, the present inventors have demonstrated for the first time a possible genetic basis for olfactory sensitivity by defming distinct segregating genetic populations based on the OR sequence data of the present invention.

The generation of such distinct genetic populations provides evidence that a strong relationship exists between sequence variability in ORs and odorant-specific olfactory threshold variability.

Thus, the present finding serve as the basis for correlating between the genotype and phenotype of olfactory perception. For example, if.an allelic variant (pseudogene) is a low frequency allele in a specific individual, it would likely correlate to a specific anosmia, since it is a low frequency functional disruption. Contrarily, a high frequency OR allele present in a specific population or subpopulation could indicate a specific hyperosmia, a high odorant sensitivity present in that particular population or subpopulation. Utilizing these and other guiding principles, the present OR typing approach can be utilized to elucidate the linkage between an individuals genotype and olfactory perception.

Elucidation of such a genotypic-phenotypic relationship can be effected by testing human volunteers from diverse ethnic groups which show clear genotypic variations in at least one, preferably several or more preferably all of the OR SPGs (allelic variants) described herein for threshold sensitivity to several odorants. The threshold sensitivities towards each odorant are expected to form a distribution in which its two ends will be determined as “hyposmic” (low sensitivity) and “hyperosmic” (high sensitivity). In addition, the functionality of the different OR SPGs will be determined in each individual by SNP genotyping in a high-throughput manner. Statistical analysis will then be used to identify significant correlation between specific odorant sensitivity and a particular OR allele, which might also indicate a specific interaction between the two molecules.

The capability of such approach to identify specific receptor-ligand interactions was recently demonstrated in the taste sense where differences in sensitivity to the bitter taste of Phenylthiocarbamide (PTC) were associated to a SNP in the taste receptor gene TAS2R38 [Kim U. K et. al, Science (299) 1221-1225 (2003)]. Similar associations between specific smell sensitivities and OR genes would shed light on the tremendous human olfactory variability and might open new commercial opportunities in the flavor and fragrance (F&F) industry.

A number of standard measurements are known in the art for testing olfactory function and evaluating threshold of odor detection. When co-applied and correlated, these methods provide a reliable measure of olfactory ability.

The Connecticut tests employ butanol threshold and odor identification. The University of Pennsylvania Smell Identification Test (UPSIT) is an odor identification test. Another test, the olfactory evoked response, is used in research centers along with odor identification tests to evaluate aberrant olfaction with relation to neurologic disease.

Butanol threshold test—The butanol threshold test used at the Connecticut Chemosensory Clinical Research Center involves a forced-choice test using an aqueous concentration of butyl alcohol in one sniff bottle and water in the other. The subject is asked to identify the bottle containing the odorant, with each nostril tested separately. After each incorrect response, the concentration of butanol is increased by a factor of 3 until the patient either achieves 5 correct responses or fails to correctly identify the bottle with 4% butanol. The detection threshold is recorded as the concentration at which the patient correctly identifies the butanol on 5 consecutive trials. The scoring relates the patient's threshold to a normal subject population.

Connecticut odor identification test—The odor identification test used at the Connecticut Chemosensory Clinical Research Center involves 10 items separately presented to each nostril in opaque jars. The items include 7 odorants, including baby powder, chocolate, cinnamon, coffee, mothballs, peanut butter, and soap. The test also includes 3 trigeminal stimulants. The subject is given a list of 20 items with the 10 stimuli and 10 other names as distractors and is asked to choose the name of the stimulus from this list. If the patient's choice is incorrect, a second chance is given to correctly identify the item. The function score is derived from the number of odorants correctly identified, and it relates the patient's performance to a normal control group's performance. The performances on the butanol threshold and the odor identification tests are averaged to determine a composite function score.

University of Pennsylvania Smell Identification Test (UPSIT)—The UPSIT involves 40 microencapsulated odors in a scratch-and-sniff format, with 4 response alternatives accompanying each odor. The subject takes the test alone, with instructions to guess if not able to identify the item. Anosmic patients tend to score at or near chance (10/40 correct). The scores are compared against sex- and age-related norms, and the results are analyzed. This test has excellent test-retest reliability.

The 3 above tests, given together and correlated, can provide a reliable measure of olfactory ability. Two more tests can be used; however, they are less reliable.

Cross-Cultural Smell Identification Test—This variant of UPSIT, which can be given in 5 minutes, was proposed for a quick measure of olfactory function. The 12-item Cross-Cultural Smell Identification Test (CC-SIT) was developed using input on the familiarity of odors in several countries, including China, Colombia, France, Germany, Italy, Japan, Russia, and Sweden. The odorants chosen include banana, chocolate, cinnamon, gasoline, lemon, onion, paint thinner, pineapple, rose, soap, smoke, and turpentine. These odorants were identified most consistently by representatives from each country. This test is an excellent alternative for measuring olfactory function when time there is a time limitation, since it is rapid and reliable. The disadvantage of this test is that its brevity limits its sensitivity in detecting subtle changes in olfactory function.

Olfactory evoked response—Olfactory evoked potentials are measured by electroencephalograrn (EEG) electrodes and an electrooculogram to standardize the patient reaction to eye movements. A visual tracking task is performed to ensure constant alertness to the task, and headphones playing white noise are worm to mask auditory clues. Either carbon dioxide (no odor but a trigeminal stimulant) or hydrogen sulfide is delivered via an olfactometer to the nose in a constantly flowing air stream. N1 is the first negative peak measured, and P2 is the second positive trough. Latencies are measured to these 2 values. It will be appreciated, though, that in patients with neurologic disease, the UPSIT revealed abnormality more frequently than olfactory evoked responses.

It will be appreciated that olfactory function can be tested using other methodologies. Tested subjects may be exposed to various odorants, including odorants from all 9 smell groups which include aromatic, fragrant, alliaceous (garlic), ambrosial (musky), hircinous (goaty), repulsive, nauseous, ethereal (fruity) and empyreumatic (roasted coffee). Such odorants are listed in Table 1, below.

It will be further appreciated that since age is known to significantly interfere with the smell sense, age differences between subjects should be taken into consideration and more preferably subjects of similar age are tested.

TABLE 1

| Chemical Name | Common Name |
|---|---|
| Acetic acid | |
| 5-Androst-16-en-3-one | |
| Benzaldehyde | benzaldehyde |
| 2,3-Butanedione | Diacetyl |
| Butanoic acid ethyl ester | Ethyl butyrate |
| 2-Butanone | Ethyl methyl ketone |
| 1,8-Cineole | Eucalyptol |
| 1,4-Diaminobutane | Putrescine |
| 3,7-Dimethyl-2,6-octadienal | Geranial, Citral A |
| (E)-3,7-Dimethyl-2,6-octadien-1-ol | Geraniol |
| Ethyl-2-methylbutanoate | |
| 4-Ethylphenol | p-Ethylphenol |
| Hexanoic acid | Caproic acid |
| Hydrogen cyanide | Hydrogen cyanide |
| Hydrogen sulfide | Hydrogen sulfide |
| 4-Hydroxy-3-methoxybenzaldehyde | Vanillin |
| 3-(4-hydroxyphenyl)-2-butanone | 3-(para-Hydroxyphenyl)-2- |
| | butanone |
| Isoamyl acetate | Isoamyl acetate |
| Isoamyl proprionate | |
| 2-Methoxy-4-(2-propenyl)pheol | Eugenol |
| 2methoxy5methylpyrazine | |
| 3-Methyl-1-butanol | Isoamyl alcohol |
| 3-Methyl-1H-indole | Skatole |
| 5-Methyl-2-(1-methylethyl)cyclo- | 1-Menthone |
| hexanone | |
| 5-Methyl-2-(1-methylethyl)phenol | Thymol |
| 2-Methyl-2-hexenoic acid | 2-Methyl-2-hexenoic acid |
| 2-Methyl-2-propanethiol | tert-Butyl mercaptan |
| 2-Methyl-5-(1-methylethenyl)-2- | l-Carvone |
| cyclohexene-1-one | |
| 2-Methyl-5-(1-methylethenyl)-2- | d-Carvone |
| cyclohexene-1-one | |
| 3-Methylbutanoic acid | Isovaleric acid |
| 2-Methylpropanal | Isobutryaldehyde |
| 3-(Methylthio)propionaldehyde | Methional |
| N,N-Dimethylmethanamine | Trimethylamine |
| 3-Phenyl-2-propenal | Cinnamaldehyde |
| 1-Phenyl-3-methyl-3-pentanol | Phenylethyl methyl ethyl carbinol |
| 2-Phenylethanol | beta-Phenyl ethyl alcohol |
| phenylisocyanide | Phenylisocyanide |
| 2-Propene-1-sulfinothioic acid | Allicin |
| S-2-propenyl ester | |
| Pyridine | Pyridine |
| 1-Pyrroline | |
| Triiodomethane | Iodoform |
| 4-(2,6,6-Trimethyl-2-cyclohexen- | alpha/beta Ionone |
| 1-yl)-3-buten-2-one and 4-(2,6,6- | |
| trimethyl-1-cyclohexen-1-yl)-3- | |
| buten-2-one | |
| w-Pentadecalactone | w-Pentadecalactone |

Acetic acid; Ethanoic acid;
Acetoin; 3-Hydroxy-2-butanone;
Acetone; 2-Propanone; Propan-2-one; Dimethyl ketone;
Acetophenone; Methyl phenyl ketone;
Acetylpyrazine; 2-Acetylpyrazine;
Acetylpyridine; 2-Acetylpyridine;
Acetylpyrrole; 2-Acetylpyrrole; Methyl pyrrolyl ketone;
Acetylthiazole; 2-Acetylthiazole;
Allyl sulfide; Diallyl sulfide;
Ambrox ®; (+)-Ambrox isomer;
Ambrox ®; (−)-Ambrox isomer; Ambroxan;
Ambrox ®; DL-Ambrox isomer; Synambran;
Amyl alcohol; 1-Pentanol; Pentyl alcohol;
Amyl butyrate; n-Pentyl butanoate; Amyl butanoate;
Anisole; Methoxybenzene;
Benzaldehyde;
Benzenethiol; Thiophenol; Phenyl mercaptan;
Benzothiazole;
Benzyl alcohol;
Bornyl acetate;
Butanethiol; 1-Butanethiol;
Butanone; 2-Butanone; Methyl ethyl ketone;
Butyl acetate; n-Butyl acetate;
Butyl alcohol; Butanol; 1-Butanol; n-Butanol;
Butyl butyrate; Butyl butanoate;
Butyl hexanoate; Butyl caproate;
Butyl isobutyrate; n-Butyl 2-methylpropanoate;
Butyl methylbutyrate; n-Butyl 2-methylbutyrate;
Butyl propionate; n-Butyl propanoate;
Butylamine; 1-Aminobutane;
Butyraldehyde; Butanal; n-Butanal;
Butyric acid; n-Butanoic acid;
Carvone; data for (−)-carvone
Caryophyllene; beta-Caryophyllene;
Citral; Geranial isomer
Citral; Neral isomer
Citronellol; (+)-Citronellol isomer
Cresol; 2-Methylphenol; o-Cresol;
Cresol; 3-Methylphenol; m-Cresol;
Cresol; 4-methylphenol; p-Cresol;
Cyclocitral; beta-Cyclocitral isomer
Damascenone; beta-Damascenone;
Damascone; alpha-Damascone;
Damascone; (+)-alpha-Damascone isomer;
Damascone; (−)-alpha-Damascone isomer;
Decadienal; trans,trans-2,4-Decadienal;
Decalactone; gamma-Decalactone; 4-Decanolide;
Decalactone; delta-Decalactone; 5-Decanolide;
Decanal; Aldehyde C-10; Decyl aldehyde;
Decanoic acid; Capric acid;
Decenal; 2-Decenal; (E)-2-Decenal;
Diacetyl; 2,3-dioxobutane;
Dimethoxyphenol; 2,6-Dimethoxyphenol; Syringol;
Dimethyl disulfide; Methyl disulfide;
Dimethyl trisulfide; Methyl trisulfide;
3,4-Dimethyl-1,2-cyclopentanedione;
3,5-Dimethyl-1,2-cyclopentanedione;
2,5-Dimethyl-4-methoxy-3(2H)-furanone
Dimethylpyrazine; 2,3-Dimethylpyrazine;
Dimethylpyrazine; 2,5-Dimethylpyrazine;
Dimethylpyrazine; 2,6-Dimethylpyrazine;
Dimethylthiazole; 4,5-Dimethylthiazole;
Dimethyltrithiolane; 3,5-Dimethyl-1,2,4-trithiolane,
Dodecalactone; gamma-Dodecalactone; 4-Dodecanolide;
Dodecalactone; delta-Dodecalactone; 5-Dodecanolide;
Dodecanal; Lauric aldehyde; Aldehyde C-12; Dodecyl aldehyde;
Dodecanoic acid, Lauric acid;
Ethoxymethylpyrazine; 2-Ethoxy-3-methylpyrazine;
Ethyl acetate;
Ethyl acetoacetate; Acetoacetic acid, ethyl ester;
Ethyl acrylate;
Ethyl alcohol;
Ethyl benzoate;
Ethyl butyrate; Ethyl butanoate;
Ethyl cinnamate;
Ethyl heptanoate;
Ethyl hexanoate; Ethyl caproate;
Ethyl isobutyrate; Ethyl 2-methylpropanoate;
Ethyl lactate;
Ethyl methylbutyrate; Ethyl 2-methylbutyrate;
Ethyl 3-methylthiopropionate;
Ethyl palmitate; Ethyl hexadecanoate; Ethyl cetylate;
Ethyl phenylacetate;
Ethyl propionate; Ethyl propanoate;
Ethyl valerate; Ethyl pentanoate;
Ethyl vanillin; 3-Ethoxy-4-hydroxybenzaldehyde; Ethavan;
2-Ethyl-3,5-dimethylpyrazine;
2-Ethyl-3,6-dimethylpyrazine;
Ethyldimethylpyrazine; 3-Ethyl-2,6-dimethylpyrazine;
Ethylguaiacol; 4-Ethylguaiacol; 4-Ethyl-2-methoxyphenol;
Ethylhexanol; 2-Ethyl-1-hexanol; 2-Ethylhexan-1-ol;
Ethylhydroxymethylfuranone; 2-Ethyl-4-hydroxy-5-methyl-3(2H)furanone;
Ethyl methoxypyrazine; 2-Ethyl-3-methoxypyrazine;
Ethyl methylpyrazine; 2-Ethyl-5-methylpyrazine;
Ethyl methylpyrazine; 3-Fthyl-2-methylpyrazine;
Ethyl methylpyridine; 5-Ethyl-2-methylpyridine;
Ethylpyrazine; 2-Ethylpyrazine;
Eucalyptol; Cineole; 1,8-Cineole; 1,8-epoxy-p-menthane;
Eugenol; 4-Allyl-2-methoxyphenol;
Eugenyl methyl ether; Methyl eugenol; Methyl eugenol ether;
Farnesol;
Formic acid;
Furfural;
Furfuryl alcohol;
Furfuryl mercaptan;
Furyl methyl ketone; 2-Furyl methyl ketone; 2-Acetylfuran;
Geraniol; trans-3,7-Dimethyl-2,7-octadien-ol;
Geranyl acetate;
Geranyl acetone; 6,10-Dimethyl-5,9-undecadien-2-one;
Geranyl isobutyrate; Geranyl 2-methylpropanoate;
Geranyl propionate; Geranyl propanoate;
Glycerol; Glycerin;
Glycine; Aminoacetic acid;
Guaiacol; o-Methoxyphenol; o-Hydroxyanisole,
Heptalactone; gamma-Heptalactone; 4-heptanolide;
Heptanal; Aldehyde C-7; Heptaldehyde; Heptyl aldehyde;
Heptanoic acid;
Heptanone; 2-Heptanone; Methyl amyl ketone;
Heptenal, 4-Heptenal (cis and trans);
Heptenal; trans-2-Heptenal;
Heptenal; (E)-4-Heptenal; trans-4-Heptenal;
Heptenal; (Z)-4-Heptenal; cis-4-Heptenal;
Heptenone; 3-Hepten-2-one; (E)-3-Hepten-2-one
Heptyl alcohol; 1-Heptanol; n-Heptanol; Alcohol C-7;
Heptyl isobutyrate; Heptyl 2-methylpropanoate;
Hexadienal; (E,E)-2,4-Hexadienal; trans,trans-2,4-Hexadienal;
Hexalactone; gamma-Hexalactone; 4-Hexanolide; Hexan-4-olide;
Hexanal; Aldehyde C-6; Caproic aldehyde;
Hexanoic acid; Caproic acid;
Hexanol; 1-Hexanol; Hexyl alcohol; Caproic alcohol; Alcohol C-6;
Hexenal; 2-hexenal; Hex-2-enal; (E)-2-hexenal;
Hexenal; cis-3-Hexenal; (Z)-3-hexenal;
Hexenol; 3-Hexen-1-ol; cis-3-Hexenol; (Z)-3-Hexenol;
Hexyl acetate;
Hexyl butyrate; Hexyl butanoate;
Hexyl isobutyrate; Hexyl 2-methylpropanoate;
Hexyl methylbutanoate; Hexyl 2-methylbutanoate;
Hexyl propionate; Hexyl propanoate;
Hydroxydecadienoic acid lactone; 6-Pentyl-alpha-pyrone,
Hydroxydihydrotheaspirane; 6-Hydroxydihydrotheaspirane;
Indole;
lonone; alpha-lonone;
lonone; beta-lonone;
Isoamyl acetate; 3-Methylbutyl acetate;
Isoamyl alcohol, 3-Methyl-1-butanol; Isopentyl alcohol;
Isobutyl acetate;
Isobutyl alcohol; 2-Methyl-I-propanol;
Isobutyl isobutyrate; 2-Methylpropyl 2-methylpropanoate;
Isobutyimethoxypyrazine; 2-Isobutyl-3-methoxypyrazine;
Isobutylmethylpyrazine; 2-Isobutyl-3-methylpyrazine;
Isobutylthiazole; 2-lsobutylthiazole;
Isobutyraldehyde; 2-Methylpropanal;
Isobutyric acid; 2-Methylpropanoic acid;
Isovaleric acid; 3-Methylbutanoic acid;
Jasmine lactone; Dec-7-en-5-olide;
Limonene; d-Limonene;
Linalool;
Maltol; Veltol; Corps praline;
5-Ethyl-3-hydroxy-4-methyl-2(5H)-furanone; Maple furanone;
Menthenethiol; 1-p-Menthene-8-thiol;
Menthone; p-Menthan-3-one;
2-Methoxy-3-isopropylpyrazine;
2-Methoxy-5-isopropylpyrazine;
Methoxymethylphenol; 2-Methoxy-4-methylphenol; Creosol;
2-Methoxy-3-sec-butylpyrazine;
2-,5 or 6-Methoxy-3-methylpyrazine;
2-Methoxy-3-methylpyrazine;
5-Methoxy-2-methylpyrazine;
Methoxypyrazine; 2-Methoxypyrazine;
Methoxyvinylphenol; 2-Methoxy-4-vinylphenol; 4-Vinylguaiacol;
Methyl butyrate; Methyl butanoate;
Methyl 2-furylmethyl disulfide;
Methyl heptanoate;
Methyl hexanoate, Methyl caproate;
Methyl isobutyrate; Methyl 2-methyl propanoate;
Methyl mercaptan;
Methyl methylbutyrate; Methyl 2-methylbutyrate;
Methyl 3-methylthiopropionate;
Methyl octanoate; Methyl caprylate;
Methyl 1-propenyl disulfide;
Methyl salicylate;
Methyl sulfide; Dimethyl sulfide; Methylthiomethane;
Methyl valerate; Methyl pentanoate;
4-Methylacetophenone; p-Methylacetophenone;
Methylbutyl acetate, 2-Methylbutyl acetate;
Methylbutyraldehyde; 2-Methylbutyraldehyde;
Methylbutyraldehyde; 3-Methylbutyraldehyde; Isovaleraldehyde;
Methylbutyric acid; 2-Methylbutyric acid;
Methylcyclopentenolone; Cyclotene; Ketonarome; Corylone; MCP;
2-Methyl-3-(furfurylthio)pyrazine
2-Methyl-5-(furfurylthio)pyrazine
Methyl heptadienone; 6-Methyl-3,5-heptadien-2-one;
Methylheptenol; 6-Methyl-5-hepten-2-ol;
Methylheptenone; 6-Methyl-5-hepten-2-one;
2-Methyl-4-propyl-1,3-oxathiane;
(+)-cis-2-Methyl-4-propyl-1,3-oxathiane;
(−)-cis-2-Methyl-4-propyl-1,3-oxathiane;
Methylpyrazine, 2-Methylpyrazine;
4-Methyl-5-thiazoleethanol; Sulfurol;
Methylthioacetaldehyde; 2-Methylthioacetaldehyde;
Methylthiomethylpyrazine (mixture of isomers);
2-Methylthio-3-methylpyrazine;
5-Methylthio-2-methylpyrazine;
Methylthiophencarboxaldehyde; 2-Formyl-5-methylthiophene;
Methylthiopropanal; 3-(Methylthio)-propanal; Methional;
Myrcene;
Myristaldehyde; Tetradecanal; Aldehyde C-14 (Myristic);
Myristic acid; Tetradecanoic acid;
Nerol;
Nonadienal; (E,Z)-2,6-Nonadienal; trans,cis-2,6-Nonadienal;
Nonadienal; (E,E)-2,4-Nonadienal; trans,trans-2,4-Nonadienal;
Nonanal; Nonyl aldehyde; Aldehyde C-9;
Nonanoic acid;
Nonanol; 1-Nonanol; Nonyl alcohol; Alcohol C-9;
Nonanone; 2-Nonanone; Methyl heptyl ketone;
Nonenal; 2-Nonenal;
Nonenal; cis-6-Nonenal;
Nootkatone; (+)-Nootkatone (the natural isomer)
Nootkatone; (−)-Nootkatone (the unnatural isomer)
Octalactone; delta-Octalactone; 5-Octanolide;
Octalactone; gamma-Octalactone; 4-octanolide,
Octanal; Caprylic aldehyde; Aldehyde C-8;
Octanoic acid; Caprylic acid;
Octanol; 1-Octanol; Octyl alcohol,- Alcohol C-8;
Octanone; 2-Octanone;
Octanone; 3-Octanone;
Octenal; 2-Octenal;
Octenol; 1-Octen-3-ol;
Octenone; 1-Octen-3-one;
Octyl acetate;
Octyl isobutyrate; Octyl 2-methylpropanoate;
Palmitic acid; Hexadecanoic acid;
Pentadecalactone; omega-Pentadecalactone; 15-Pentadecanolide;
Pentanone; 2-Pentanone;
Pentenal; 2-Pentenal;
Pentenol, 1-Penten-3-ol;
Pentenone, 1-Penten-3-one; Ethyl vinyl ketone;
Pentenone, 3-Penten-2-one;
Pentylfuran; 2-Pentylfuran;
Pentylpyridine; 2-Pentylpyridine,
Phenethyl alcohol; 2-Phenethyl alcohol;
Phenol;
Phenylacetaldehyde;
Phenylacetic acid;
Pinene; alpha-Pinene;
Pinene; beta-Pinene;
Piperidine;
Piperonal; Heliotropine;
Propanethiol; 1-Propanethiol; n-Propyl mercaptan;
Propanol; 1-Propanol; Propyl alcohol;
Propenyl propyl disulfide; Propyl propenyl disulfide;
Propenylguaethol; 2-Ethoxy-5-propenylphenol;
Propionaldehyde; Propanal;
Propionic acid; Propanoic acid;
Propyl butyrate; Propyl butanoate;
Propyl propionate; Propyl propanoate;
Pyrazinyl methyl sulfide; 2-(Methylthiomethyl)-pyrazine;
Pyridine;
Pyrrole;
Pyrrolidine;
Quinoline;
Raspberry Ketone; 4-(p-Hydroxyphenyl)-2-butanone; Oxanone,
Rose oxide; 4-Methyl-2-(2-methylpropen-1-yl)-tetrahydropyran;
Sinensal; alpha-Sinensal;
Sotolon; Caramel furanone;
Stearic acid; Octadecanoic acid;
Strawberry furanone; 4,5-Dimethyl-3-hydroxy-2(5H)-furanone;
Styrene; Vinylbenzene;
Terpineol; alpha-Terpineol; p-Menth-1-en-8-ol;
TerPinolene; p-Menth-1,4(8)-diene;
Tetramethylpyrazine, 2,3,5,6-Tetramethylpyrazine;
Thiamine hydrochloride; (value for thiamine pure)
Thymol, 5-Methyl-2-isopropylphenol;
Trimethylamine;
2,2,6-Trimethylcyclohexanone,
Trimethyloxazoline; 2,4,5-Trimethyl-3-oxazoline;
Trimethyl pyrazine; 2,3,5-Trimethylpyrazine,
Trimethylthiazole; 2,4,5-Trimethylthiazole;
Undecalactone; delta-Undecalactone; 5-Undecanolide;
Undecanal; Aldehyde C-11 (undecylic);
Undecanoic acid; Undecylic acid;
Unclecanone; 2-Undecanone; Methyl nonyl ketone;
Valeraldehyde; Pentanal;
Valeric acid; Pentanoic acid;
Vanillin, 4-Hydroxy-3-methoxybenzaldehyde;
Vinylphenol; 4-Vinylphenol;

Once a correlation between an individuals (or population) genotype and phenotype is established, such a correlation can be used to match fragrance and flavors to specific consumers, consumer groups, subpopulations and populations (pharmacogenomics of olfaction). Moreover, identification of specific receptor-odorant interactions can offer ways to more efficiently design and deliver pleasing scents and flavors. Technologies for modulating odor response can utilize information uncovered using the present methodology to alter the olfactory response by molecular means (receptors agonists and antagonists) or to block or enhance the perception of specific smells.

In addition to being usefull in olfactory typing, the OR alleles of present invention and information derived therefrom can also be utilized in fertility testing.

Recent studies have shown that several disctinct ORs are also predominantly or exclusively expressed in human spermatogenic cells [Parmentier (1992) Nature 355:453; Vanderhaeghen (1993) J. Cell Biol. 123:1441]. Immunocytochemistry indicates that resepctor proteins are localized to the sperm flagellar midpiece [Vanderhaeghen (1993) J. Cell Biol. 123:1441]. These observations have led to speculation that ORs may also function in chemosensory signaling pathways and hence in direct sperm chemotaxis.

Recently Spehr and co-workers have cloned and functionally expressed a testicular OR, termed hOR17-4. Spehr and his colleagues exposed hOR17-4 to a number of different chemicals to determine which activated this protein. This study uncovered that in the presence of bourgeonal, some human sperm became activated and began to move toward the source of the chemical, indicating that ORs may also govern sperm movement towards the egg. Spehr noted that researchers have discovered between 20 and 40 olfactory receptors which, like hOR17-4, are localized to testicular tissue.

In view of the above findings, it is highly likely that the olignucleotides and/or antibodies of the present invention can be used to detect/diagnose male infertility, particularly that associated with deficiency in sperm motility or to detect specific odorants which may suppress or enahnce sperm motility. Only recently has it been shown that sperm motility parameters are important for both presenting the maximum number of male gametes to the egg as well as facilitating penetration through its zona pellucida. Sperm motility parameters were also found to have high correlation with fertilization rates in vitro [Mahadevan and Trounson (1984) Fertil. Steril. 24: 131-4].

Detecting or diagnosing male infertility is typically effected using a sperm sample (i.e., semen) obtained from the subject tested. Semen can be collected by any method which is generally used for that species. For example, bovine and rabbit semen is typically collected by use of an artificial vagina. Human semen is typically collected by manual ejaculation. Methods of extracting proteins or nucleic acids from biological samples and methods of probing such samples with the oligonucleotides and/or antibodies of the present invention are described hereinabove.

To facilitate diagnosis the oligonucleotides and antibodies generated according to the teachings of the present invention can be included in a diagnostic kit. These reagents can be packaged in a one or more containers with appropriate buffers and preservatives and used for diagnosis.

Preferably, the containers include a label. Suitable containers include, for example, bottles, vials, syringes, and test tubes. The containers may be formed from a variety of materials such as glass or plastic.

In addition, other additives such as stabilizers, buffers, blockers and the like may also be added.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif.(1990); Marshak et al., “Strategies for Protein Purification and Characterization—A Laboratory Course Manual” CSHL Press (1996); “Approaches to Gene Mapping in Complex Human Diseases” Jonathan L. Haines and Margaret A. Pericak-Vance eds., Wiley-Liss (1998); “Genetic Dissection of Complex Traits” D. C. Rao and Michael A. Province eds., Academic Press (1999); “Introduction to Quantitative Genetics” D. S. Falconer and Trudy F. C. Mackay, Addison Wesley Longman Limited (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

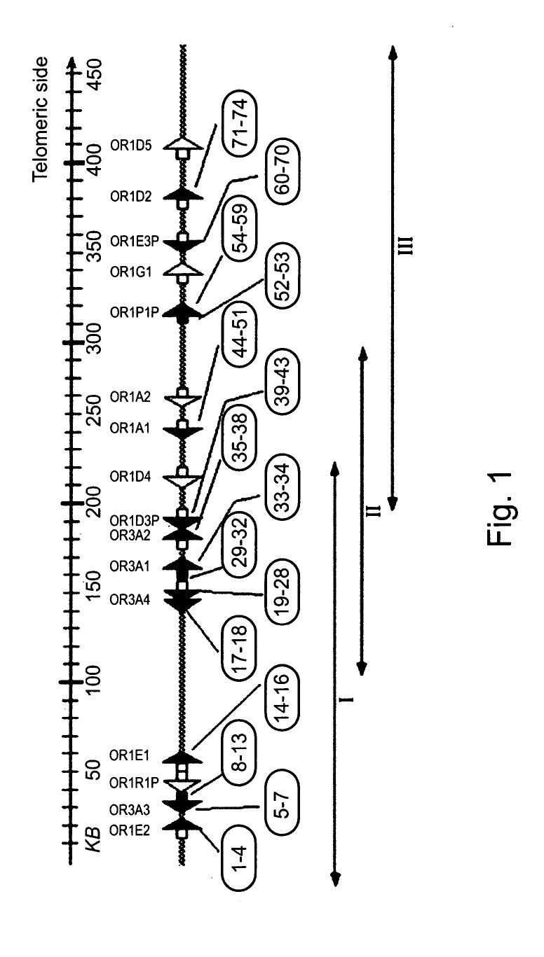

Population samples and DNA sequencing -12 olfactory receptor (OR) coding regions and 3 OR introns (1000 bp each) scattered along ˜400 Kb of the OR gene cluster (FIG. 1) on human chromosome 17p13.3 (15 Kb in total) were subjected to PCR amplification from 35 unrelated individuals consisting of 4 different ethnogeographic populations: 10 Ashkenazi Jews, 10 Yemenite Jews, 8 Bedouins, and 7 Pygmies.

PCR amplification—PCR reactions were carried out in a volume of 25 μl, containing 0.2 μM of each deoxynucleotide (Promega Corp., Madison, Wis., USA), 50 pMol of each primer, PCR buffer containing 1.5 □M MgCl₂, 50 [□M KCl, 10 □M Tris-HCl pH 8.3, one unit of Taq DNA polymerase (Boehringer Mannheim, Germany) and 50 ng of genomic DNA. PCR reactions included an initial denaturation step of 3 minutes at 94° C., followed by 35 cycles of denaturation (1 minute at 94° C.), annealing (I minute) at either 55° C. or 60° C., extension (1 minute at 72° C.) and a final extension step of 10 minutes at 72° C. PCR products were subjected to 1% agarose gel electrophoresis, and were further purified using the High Pure PCR Product Purification Kit (Boehringer Mannheim, Germany).

Sequencing analysis—Purified PCR products were subjected to dye-terminator cycle sequencing reactions (Perkin Elmer, Boston, Mass., USA). Extension reactions were electrophoresed on an ABI 3700 automated DNA sequencer (Applied Biosystems, Foster City, Calif. USA). After base calling with the ABI Analysis Software (version 3.0), the analyzed data was edited using the Sequencher program (version 4.0, GeneCodes Corp., Ann Arbor, Mich., USA).

For each individual, a genomic segment of an approximately 1 kb which was sequenced from both ends was assembled using the Sequencher software to identify DNA polymorphisms. Sequencing was repeated for each genomic segment containing a singleton.

Statistical analysis—Summary of the nucleotide variability was calculated for each ethnogeographic group using the Watterson's theta (d,) (Watterson, 1975), which is based on the number of segregating sites in the sample, and the nucleotide diversity π (Nei and Li, 1979), which is the average number of differences between all pairs of sequences- in the sample. The Tajima's D-test (Tajima, 1993#was used to estimate whether the frequency spectrum of alleles deviates significantly from the expectations of a standard neutral model. Tajima's D is positive in case of an excess of intermediate frequency alleles, and negative in case of an excess of rare alleles. Positive Tajima's D values may be caused by recent bottlenecks or balancing selection, while negative Tajima's D values may be caused by recent selective sweep, purifying selection or population expansion.

Haplotype inference—The sequencing data reflected individuals heterozygotes for multiple DNA polymorphisms with ambiguous haplotype structure. To resolve the haplotype structure of each sample the Clark's haplotype subtraction algorithm (Clark, 1990) was employed. The rationale of this algorithm is that homozygous haplotypes are probably common and that a double heterozygote is likely to contain known common haplotypes. The Clark's algorithm is composed of three steps: 1) Identification of all unambiguous haplotypes (all homozygous and sequences with one heterozygous site) and considering them as ‘resolved’. 2) Determination of whether each of the resolved haplotypes could be one of the alleles in the remaining ambiguous sequences. 3) In each case a possible phase of a double heterozygote is identified as one of the resolved ones, the phase is assumed to be known, and the remaining haplotype is added to the resolved haplotype set. This algorithm has been previously used and, in particular cases, has proven to be reliable by comparison with haplotypes obtained by direct molecular methods (Clark et al., 1998; Rieder et al., 1999).

To reveal the population substructure and patterns of linkage disequilibrium (LD), all rare variants (<0.15) were excluded from the data before applying the algorithm. Then the algorithm was applied separately to the three major parts of the cluster (see FIG. 1), allowing 6-12 overlapping SNPs between each pair of segments. Once all the haplotypes were resolved, the full-length haplotype was constructed using the overlapping SNPs and the subsets of the heterozygous sites were compared with the experimentally determined haplotype information of Gilad et al. (2000).

Population stratification—The nearest neighbor statistic (Sₙₙ, Hudson, 2000) was used to test for population substructure. This method is a measure of how often a pair of nearest haplotypes (based on sequence similarity) belongs to the same ethnogeographical population group. The Sₙₙ value approaches unity when the populations at the two localities are highly differentiated and is 0.5 when the populations are part of the same panmictic population (Hudson, 2000). A permutation test is used to assess whether the Sₙₙ is significantly large for a particular sample, indicating that the populations at the two localities are differentiated. For genotype data in a small number of individuals with extensive recombination, this method was shown to perform better than alternative ones (Hudson, 2000). The commonly used Fₛₜ statistic (Wright, 195 ) was also calculated for all pairwise population groups.

LD and recombination—The coefficient D′ (Lewontin, 1964) was used as a measure of LD between polymorphic sites, using the Graphical Overview of Linkage Disequilibrium (GOLD) software (Abecasis and Cookson, 2000), applying a Fisher exact test (FET) for statistical significance. An alternative method used for pairwise LD computation was the Expectation Maximization (EM) algorithm (Excoffier, 1995) using the Arlequin software (http://lgb.unige.ch/arlequin).

### Example 1

### Population—Specific Nucleotide Diversity within the Human Olfactory Receptor (OR) Gene Cluster

The olfactory receptor gene cluster on human chromosome 17p13.3 was studied using SNP analysis in four distinct ethnogeographic populations: Ashkenazi Jews, Yemenite Jews, Bedouins and Pygmies.

Experimental and Statistical Results

Identification of SNPs within the OR gene cluster—SNP scoring was performed by sequencing of 12 OR coding regions and segments within three OR introns of 35 unrelated individuals from four disparate ethnogeographic origins: 10 Ashkenazi Jews, 10 Yemenite Jews, 8 Bedouins, and 7 Pygmies. Genotyping was employed for each individual along the entire OR cluster.

A total of 74 polymorphic sites were found, of which 31 were novel (http://bioinfo.weizmann.ac.i1/˜menashe/OR17_SNPs.html). Two of the SNPs identified, in pseudogenes OR1P1P and OR1E3P segregated between the pseudogenized and intact forms. Noteworthy, the variability within the same two ORs displayed significant deviations from Hardy-Weinberg equilibrium, whereby 7 out of 10 SNPs (singletons excluded) were not at equilibrium (Table 2).

TABLE 2
Sequence diversity and Neutrality test for the OR
gene cluster on Chromosome 17p13.3

| | | | | | | Tajima's | P value (of |
|---|---|---|---|---|---|---|---|
| | n^{a} | S^{b} | Singletons | π (%) | θ_{w} (%) | D | Tajima D) |
| Total | 35 | 74 | 19 | 0.12 | 0.10 | 0.42 | >0.1 |
| Pygmies | 7 | 57 | 8 | 0.14 | 0.12 | 0.88 | >0.1 |
| Bedouin | 8 | 55 | 6 | 0.12 | 0.12 | 0.12 | >0.1 |
| Yemenite Jews | 10 | 46 | 4 | 0.10 | 0.09 | 0.51 | >0.1 |
| Ashkenazi Jews | 10 | 46 | 1 | 0.08 | 0.08 | −0.32 | >0.1 |
| Average | 8.75 | 51 | 4.75 | 0.11 | 0.10 | 0.30 | |
| SD | 1.50 | 5.83 | 2.99 | 0.03 | 0.02 | 0.52 | |

Population variability values are given for the total data set and for each population group separately.
π is the nucleotide diversity,
θ is the population mutation rate.
P values for Tajima's D are calculated using DnaSP v. 3.12.
n^{a} = sample size.
S^{b} = number of SNPs.

Nucleotide diversity—Distinct differences were seen among the human populations. The highest nucleotide diversity was found in the Pygmy population (π=0.14%, Table 2), while the lowest value was in the Ashkenazi Jews (π=0.08%, Table 1). Consequently the singletons were unequally distributed, with as many as 8 in the African Pygmies and only 1 in the Ashkenazi Jews. These values are consistent with a historically small population size for Ashkenazi Jews and with the previously reported high variability in Africans (Hammer et al., 2000; Kobyliansky et al., 1982; Przeworski et al., 2000).

SNPs frequencies do not deviate from the neutral model—The overall values of θ_{w}=15.2 (0.10% per bp) and π=0.12% were found to be within the range previously reported for the presently studied cluster (Gilad et al., 2000) and elsewhere (Clark et al., 1998; Fullerton et al., 2000; Subrahmanyan et al., 2001). The overall θ_{w} was not significantly different from 19, the observed number of SNPs seen only once in the sample (singletons) (T-test, P=0.36), as predicted by neutral expectations.

The Tajima's D statistic (Tajima, 1989) was computed to compare the observed frequency spectrum of SNPs to neutral model expectations (Table 2). The values for the entire data set, as well as for the individual populations, did not represent a statistically significant deviation from neutrality (P values>0.1 in all cases, Table 2) although the Tajima's D values of the Pygmy population (D=0.88) and the Ashkenazi Jewish population (D=−0.32) present two extremes.

Altogether, these data uncovered 31 new SNPs and confirmed 43 known SNPs within the OR gene cluster on chromosome 17p13.3. These findings demonstrate distinct nucleotide diversities between the four different ethnogeographic groups with the highest value found in the African Pygmy population and the lowest value found in the Ashkenazi Jewish population. However, the observed frequency spectrum of SNPs did not represent a statistically significant deviation from neutrality. These results demonstrate a highly documented SNP mapping within the OR gene cluster on chromosome 17p13.3 which can be used for haplotype analysis and linkage disequilibrium determination.

### Example 2

### Linkage Disequilibrium along the Olfactory Receptor (OR) Gene Cluster on Chromosome 17p13.3

To calculate the level of linkage disequilibribium (LD) along the OR gene cluster within the four ethnogeographic groups the various haplotypes and recombination events were calculated.

Experimental and Statistic Results

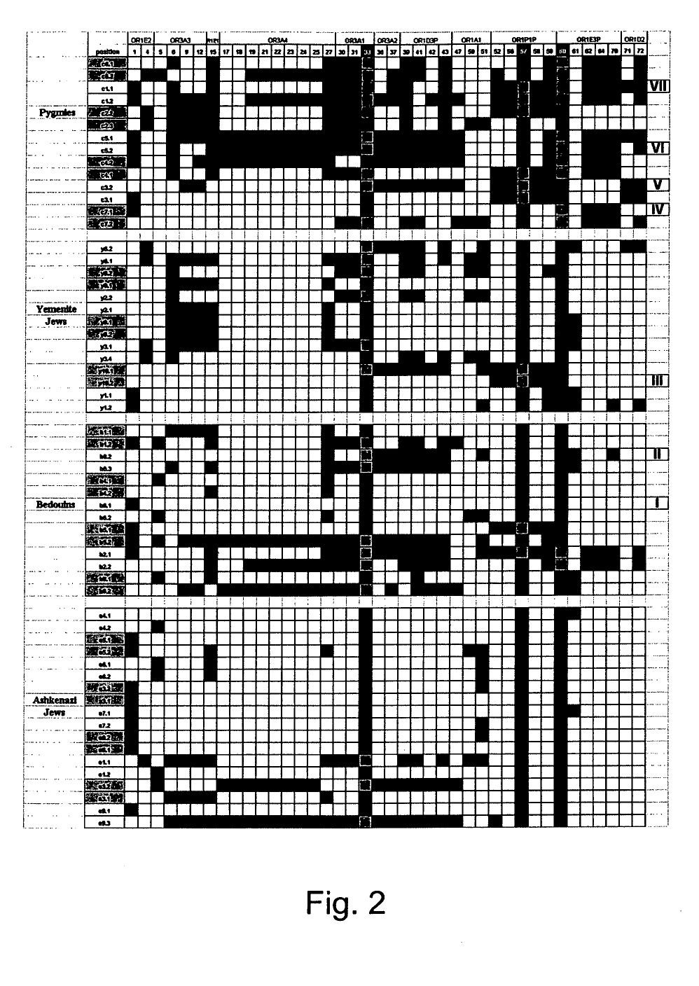

Haplotype reconstruction—To calculate the LD, 40 polymorphic sites with intermediate frequencies (higher than 0.15) were subjected to haplotype re-construction using the Clark's algorithm as described in Methods hereinabove. Using this algorithm, 47 haplotypes from 30 individuals were successfully elucidated (FIG. 2). However, the algorithm failed to resolve the haplotypes of five individuals (14%) due to ambiguities in their genotypes. However, this un-resolved haplotype fraction is not unexpected for the sample size used (Clark, 1990). Moreover, since no excess of heterozygous genotypes was seen (http://bioinfo.weizmann.ac.i1/menashe/OR17_SNPs.html) these results probably do not reflect non-specific PCR amplifications of two highly similar ORs.

Assuming neutrality, where haplotype frequency is governed only by genetic drift, and no recombination, the expected mean number of haplotypes for the observed variability values is 24 (Ewens, 1982). Therefore, the observed 47 haplotypes within 30 individuals may reflect multiple recombination events in this gene cluster. Using the DnaSP package (Rozas and Rozas, 1995), the minimal number of recombination events (Rₘ) (Hudson and Kaplan, 1985) was calculated, assuming no recurrent mutations, and was found to be 18.

Population subdivision—The haplotype distribution of the whole sample revealed a notable differentiation between the four ethnogeographic groups. As is shown in FIG. 2, while most of the haplotypes composed of rare variants are found within the Pygmy population, the majority of segregating pseudogenes with increasing disruption level are found in the Ashkenazi Jewish population.

The level of genetic differentiation of the subpopulations was further evaluated by applying the nearest-neighbor statistic (Sₙₙ) (Hudson, 2000) to the haplotype data. According to this statistic; the Sₙₙ is expected to be near one when the populations at the two localities are highly differentiated and near one half when the populations at the two localities are part of the same panmictic population. As is shown in Table 3, the highest Sₙₙ value is observed when the Pygmy population is compared with the Ashkenazi Jewish population, reflecting that these two populations are well differentiated. On the other hand, the Sₙₙ values observed when the Bedouin population is compared with the two Jewish populations (i.e., the Yemenite Jews and the Ashkenazi Jews) indicate at this particular OR cluster that the two Jewish populations are genetically closer to the Bedouins than to each other. These findings appear to correlate with the respective geographic distances, since the Eastern Mediterranean Bedouins are intermediates between the European Ashkenazi and the South Arabian Yemenites.

TABLE 3
Population subdivision: Sₙₙ test

| | Yemenite | | Ashkenazi | |
|---|---|---|---|---|
| | Jews | Bedouin | Jews | All |
| Pygmies | 0.77 | 0.79 (P < 0.01) | 0.90 (P < 0.01) | 0.86 (P < 0.01) |
| | (P < 0.01) | | | |
| | Yemenite | 0.73 (P = 0.01) | 0.83 (P < 0.01) | 0.82 (P < 0.01) |
| | Jews | | | |
| | | Bedouin | 0.69 (P = 0.02) | 0.74 (P = 0.02) |
| | | | Ashkenazi | 0.79 (P < 0.01) |
| | | | Jews | |

The nearest neighbor statistic values are given for the total dataset (All) and for pairwise group comparisons with the corresponding p values.

The population substructure was further evaluated using the Fₛₜ statistic (Wright, 1951). As is shown in Table 4 hereinbelow, the four ethnogeographic populations did not exhibit a significant differentiation from each other. This is probably due to the fact that the Fₛₜ test has low power when small populations with a substantial recombination rate are compared. However, as with the Sₙₙ test, the comparison of the Pygmy population with the Ashkenazi Jewish population displayed the highest Fₛₜ value, demonstrating that these two populations are highly differentiated from each other.

TABLE 4
Population subdivision; Fₛₜ test

| | Yemenite Jews | Bedouin | Ashkenazi Jews | All |
|---|---|---|---|---|
| Pygmies | 0.17 | 0.12 | 0.27 | 0.19 |
| | Yemenite Jews | 0.01 | 0.03 | 0.02 |
| | | Bedouin | 0.04 | 0.00 |
| | | | Ashkenazi Jews | 0.08 |

The Fₛₜ values are given for the total dataset (All) and for pairwise group comparisons.

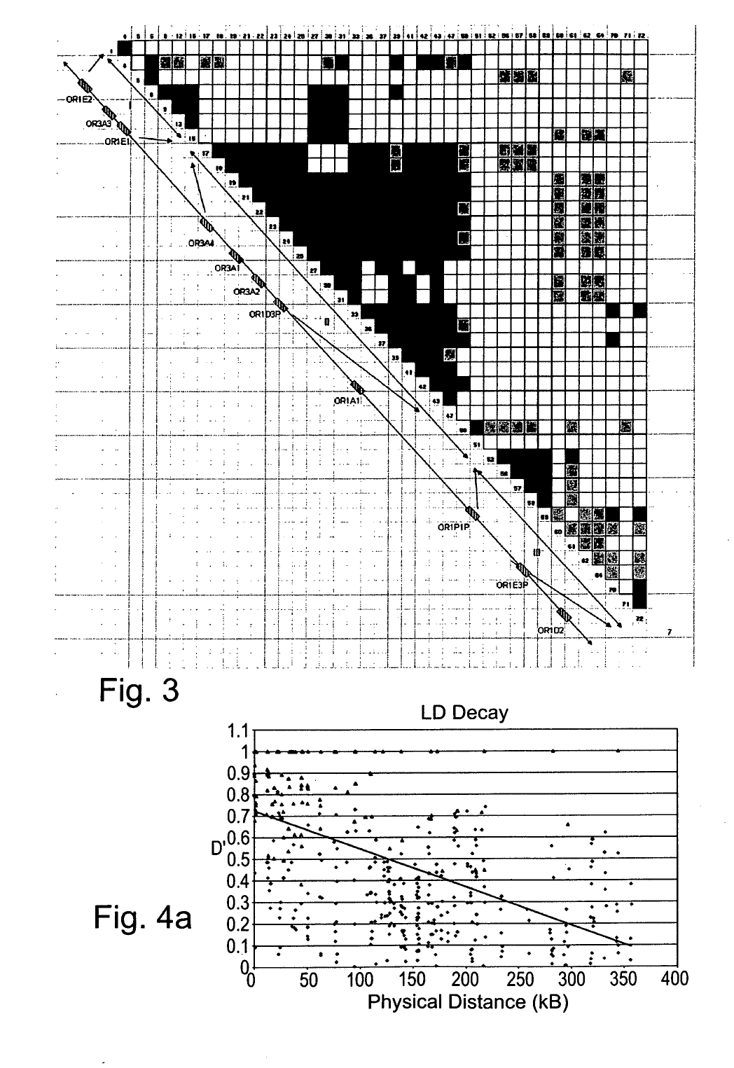

Determination of linkage disequilibrium (LD)—Based on the inferred haplotype information, the level of linkage disequilibrium, D′ (Lewontin, 1964) was calculated for all pairs of sites in the sample. As is shown in FIG. 3, SNPs scattered along the entire three regions within the OR gene cluster were aligned according to their physical order and the level of LD between them was determined. Since the Clark's method tends to reduce the number of haplotypes and thus may inflate the estimate of LD, two different statistic tests were employed to assess the significance of the LD. These are the Fisher Exact Test (FET) and the Expectation Maximization (EM) algorithm (Excoffier, 1995). Both tests were applied directly to the genotype data, using the same frequency cutoff and the same 30 individuals. As is further shown in FIG. 3, the similarity between the two algorithms was extremely high for the first two regions of the cluster (regions I and II), with respectively 94.3% and 98.6% of the results found to be in agreement. In the third region, the concordance level was only 64.8%. In this case, the haplotype-based method is preferred. The poor performance of the EM algorithm in this region is probably due to deviations from Hardy-Weinberg equilibrium in the two OR pseudogenes in this region. The inferiority of the direct method in this region is revealed through discrepancies in intragenic haplotypes as previously determined using cloning of PCR products (Gilad et al., 2000).

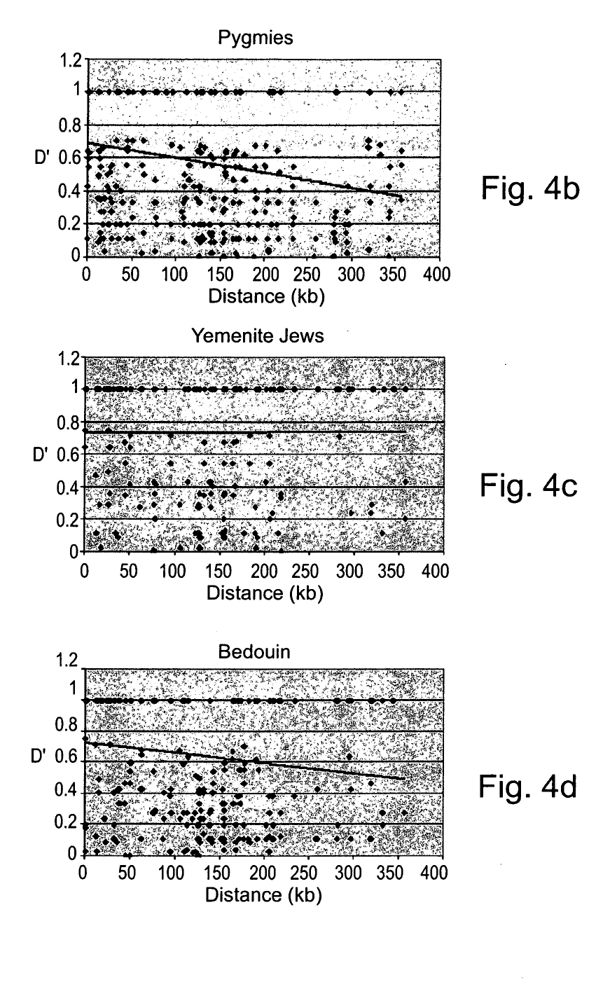

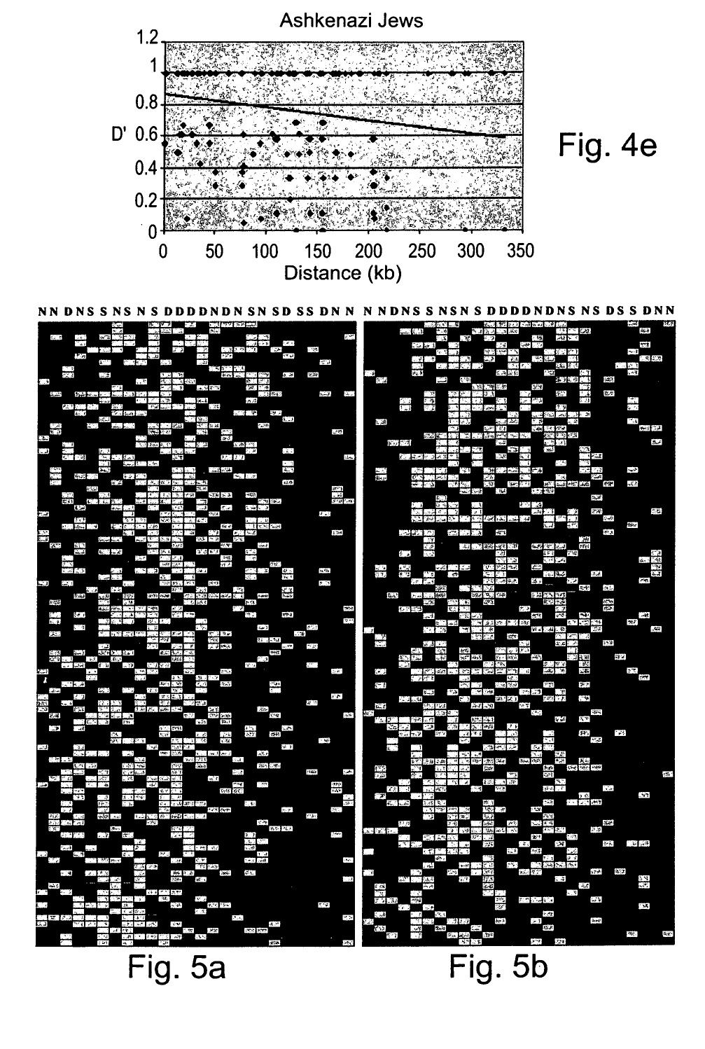

Determination of LD decay along the entire OR gene cluster on chromosome 17p13.3—To evaluate the LD decay along the entire OR region the D′ values were plotted against pairwise physical distance. As is shown in FIG. 4a, the LD decay to D′=0.5 for the entire dataset was seen at a distance of 124 Kb (R=−0.53, P<0.01). However, since the entire dataset was composed of four highly differentiated populations, the LD decay was further analyzed and compared between the four ethnogeographic groups. These analyses revealed significant decays to D′=0.5 for the Pygmy population at a distance of 210 Kb (R=−0.24, P<0.01, FIG. 4b). On the other hand, the Bedouins and both Jewish populations exhibited high level of LI) with no evidence of decay at distances of 320 Kb (R=−0.16, p<0.01, FIG. 4d), 400 Kb (R=−0.11, p=0.08, FIG. e) and 1.1 Mb (R=−0.01, P=0.41, FIG. 4c) for the Bedouins, Ashkenazi Jews and Yemenite Jews, respectively. The minimal recombination values varied roughly in accordance and were Rₘ32 12, 11, 5 and 6 for the Pygmies, Bedouins, Yemenite Jews and Ashkenazi Jews. In addition, the four ethnogeographic groups exhibited different distribution of LD across regions (data not shown). However, given the small sample sizes, these differences in spatial patterns of LD may only be taken as indicative and not statistically proven.

Altogether these results demonstrate that the OR gene cluster on chromosome 17p13.3 exhibits a particularly slow decay of LD, whereby D′ decreases to 0.5 at an average distance of 124 Kb, and no decay is observed when the Pygmies are omitted from the analysis. These results support previous findings demonstrating a significantly higher level of LD in non-African samples (Reich et al., 2001; Frisse et al., 2001).

Therefore, these results constitute a solid basis for an association study between genetic variations within the OR gene cluster on human chromosome 17p13.3 and specific olfactory phenotypes. The conspicuous population substructure and the long stretches with significant LD found within these populations strongly suggest the use of only a few markers within this cluster for the study of this kind.

### Example 3

Sequencing of five olfactory receptor pseudogenes revealed that three of the analyzed OR pseudogenes segregate between intact and disrupted forms.

Experimental and Statistic Results

Segregating pseudogenes in the entire sample population—The sequence analysis included five OR pseudogenes. Two of which (OR1E3P and OR1P1P) have an open reading frame interrupted at only one position and leading to a potentially inactive olfactory receptor. The coding region of OR1E3P is interrupted by a single base deletion (nucleotide coordinate 54 of SEQ ID NO: 84; see, Tables 5 and 6) that causes a frame shift and results in a premature stop codon. The coding region of OR1P1P is interrupted by a nonsense mutation (T→A at nucleotide coordinate 553 of SEQ ID NO: 85; see, Tables 5 and 6). Sequencing of 35 individuals from the four ethnogeographic groups (Pygmies, Bedouins, Yemenite Jews and Ashkenazi Jews) revealed that these two mutations in OR coding regions were polymorphic in the entire sample. The single base deletion of OR1E3P (causing a truncated protein) was absent in 12/70 (17%) of the chromosomes, and the nonsense mutation in OR1P1P was not seen in 14/70 (20%/o) of the chromosomes.

A third pseudogene, OR3A1, has a single nucleotide substitution (G→A at coordinate 374 of SEQ ID NO: 89; see, Tables 5 and 6) which yields an amino acid replacement (Arginine 125 to Glutamine) in the DRY motif, a highly conserved position in ORs and other G-protein-coupled receptors (GPCRs) (Probst et al., 1992). This residue has been suggested to play a crucial role in signaling-related conformational changes in 7-helix receptors (Alewijnse et al., 2000). Therefore, this mis-sense mutation (R125Q) may reflect a third pseudogene, potentially leading to receptor inactivation. The mis-sense mutation was found to segregate within the entire sample population whereas the intact R125 form present in 48% of all chromosomes.

Population—specific segregating pseudogenes—When the fraction of segregating pseudogenes was analyzed and compared between the different ethnogeographic groups, unequal disposition of all three segregating pseudogenes was found (FIG. 2). In all cases, the intact form of these genes was significantly more frequent in individuals of Pygmy origin than in individuals from other origins (FIG. 2). Inspection of the haplotypes for these three SNPs revealed 7 out of the expected 8 haplotypes. Four of the chromosomes contained the all -intact haplotype, including one homozygote pygmy individual. As is further shown in FIG. 2, the more intact haplotypes (labeled in green) were found among the Pygmies, whereas, the more disrupted haplotypes (marked in red) were found in the Yemenite Jews, Bedouins and Ashkenazi Jews. The highest level of haplotypes disruption was found in the Ashkenazi Jews, where 84% of the haplotypes were completely damaged (FIG. 2).

These results demonstrate three putative SNP-related functionally segregating pseudogenes. It is likely that a functional loss at a given OR locus results from a homozygous state of the pseudogene, i.e., that olfactory dysfunction is a recessive trait (Whissell-Buechy and Amoore, 1973). Thus, individuals carrying at least one intact gene variant may have an extended olfactory ability compared to individuals who carry two pseudogene variants in the same locus. Accordingly, ten of the thirty individuals in FIG. 2 might be hyposmic or anosmic in relation to holding both disrupted alleles in all three segregating OR loci. At the other extreme, three members of the sample are expected to be functionally intact at all three loci. Other individuals constitute intermediate cases with one or two potential functional disruptions. These findings therefore suggest that different human individuals have different chemosensory repertoires.

Altogether, these results further suggest the use of these OR pseudogenes in a genotype-phenotype correlation study.

### Example 4

### Characterization of Additional Novel Polymorphic Olfactory Receptor Genes

A large-scale SNP mapping of the olfactory receptor loci was performed in 189 individuals from different ethnic origins.

Materials and Methods

DNA samples—Human genomic DNA was obtained from 28 unrelated anonymous individuals who donated blood at the Israeli Blood Bank, 30 unrelated individuals, provided by the National Laboratory for Genetics of Israeli Population at Tel Aviv University (10 Ashkenazi Jews, 10 Bedouins, 10 Yemenite Jews) and 131 samples provided by the Coriell Cell Repositories, Camden, N.J. The latter consisted of 9 Aboriginal Taiwanese, 7 Russians, 5 Mexicans, 5 Brazilians, 6 Asians and 99 African American. Genomic DNA from two chimpanzees (Pan troglodytes) was isolated from whole blood.

OR loci and SNPs selection—A consensus sequence for each OR family was generated using an alignment of 1227 intact OR sequences from human and mouse. These sequences were used as queries in tBLASTN searches against the Celera's human refSNP database. Each SNP was associated with a location within a particular OR by requiring that it resides on the same chromosome and that at least 33 amino acids from both sides of the SNP match exactly to the gene. SNPs were selected only if they unambiguously assigned to one intact OR gene and changed the open reading frame.

Sequencing and SNP genotyping—DNA sequencing was performed on PCR products as described in the General Materials and Methods for Examples 1-3 hereinabove. SNP genotyping employed the high-throughput mass-spectrometry SNP scoring system (Sequenom, San Diego, Calif., USA). The segregating OR loci were validated by repeating their genotyping in all individuals.

Experimental and Statistics Results

Screening for potential segregating pseudogenes (SPGs) in the olfactory receptor (OR) loci—Potential SPGs were screened using two approaches. In the first approach, OR pseudogenes that contain only one open reading frame disruption were included (Glusman et al., 2001). Fifty of these ORs were sequenced in chimpanzee, in which 33 of 50 were found to be intact suggesting that these sequences recently emerged in humans and thus further proving that these sequences are SPGs. In the second approach, the Celera's human single nucleotide polymorphism (SNP) database was searched for variations with potential to affect protein integrity. These included in-frame stop codons and mis-sense mutations in highly conserved amino acids with a potential to modify the olfactory receptor function (Schoneberg et al., 2002). This led to the identification of another 18 candidate segregating pseudogenes.

Identification of SPGs among 189 ethnically diverse individuals—A total of 51 OR loci were genotyped from 189 ethnically diverse individuals as detailed in Materials and Methods hereinabove. Twenty six of these ORs (Table 5) were found to segregate between the intact and disrupted alleles in the entire sample set resulting in a unique genotypic pattern for each of the tested individuals. FIGS. 5a-b summarize the various genotypic patterns of the segregating pseudogenes within the African-American (FIG. 5a) and the non-African (FIG. 5b) populations. Assuming that olfactory dysfunction is a recessive trait (Whissell-Buechy and Amoore, 1973), a large diversity in the putative phenotypes can be deduced from the different genotypes.

Altogether, a total of 178 putative phenotypes can be seen in the 189 individuals studied. Such a high level of documented inter-individual variability in a gene family is unprecedented, except in the case of the major histocompatibility complex (Yeager and Hughes, 1999).

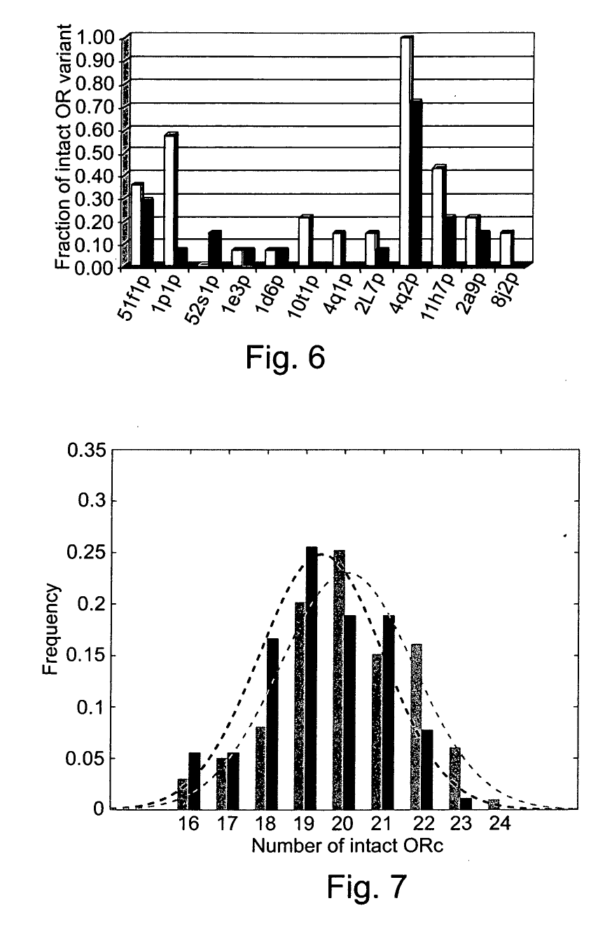

Non-Africans have significantly fewer intact ORs than African-Americans—The frequency of twelve intact OR alleles was compared between the Caucasian and Pygmy populations (FIG. 6). The frequency of the null allele for two SPGs was equal in the two populations. (OR1E3p and OrqD6p). For nine out of the other 10 SPGs, the null allele was found to be at a higher frequency in Caucasians than in Pygmies (FIG. 6, two-tailed sign test, P<0.021). Altogether, the frequencies of the null alleles were 13% higher in the Caucasian population than in the Pygmy population.

A further analysis incorporating all 26 SPGs of the present invention (listed in Table 5) revealed that the frequency of genomes having fewer numbers of intact OR (i.e., having more disrupted pseudogenes) was higher among the non-African population (FIG. 7). These results are consistent with previous results presented in Example 3 hereinabove, demonstrating a higher fraction of intact OR genes among the Pygmies as compared with the Bedouins, Yemenite Jews and Ashkenazi Jews.

Computation of the number of OR SPGs in the entire genome—The number of OR SPGs in the entire human genome was computed for 189 individuals by a two-step procedure. In the first step, based on the discovery of 11 OR SPGs among 50 singly disrupted pseudogenes tested, it was estimated that for the total of 67 such genes in the HORDE database (http://bioinformatics.weizmann.ac.i1/HORDED) there would be a proportionate number of 15 SPGs genome-wide. The assumption was that OR pseudogenes with more than one disrupting mutation are unlikely to harbor SPGs. In the second step, based on a sequencing depth of five chromosomes in the Celera SNP database (Venter et al., 2001), and assuming a neutral frequency spectrum for the disrupted derived alleles, the SPG count of 15 in 5 chromosomes extrapolates to 45 SPGs in 378 chromosomes (189 individuals). Thus, a total of 60 SPGs was computed (i.e., 15+45). Using the same formulation, the count of SPGs with a frequency higher than 1% was found to be 48.

These numbers are in rough agreement with the reported count of different modes of specific anosmias, human odorant-specific sensory deficits (Amoore, 1974).

Thus, the genotypic differences presented hereinabove might underlie at least some of the reported human phenotypic olfactory variations. Therefore, these results demonstrate the potential use of the OR SPGs for future association studies between individual OR disruptions and defined cases of odorant-specific olfactory threshold variability.

Table 6 below lists oligonucleotide sequences which can be used to establish an odorant genotype for individuals or populations thus putatively classifying such individuals or populations according to their odorant sensitivity.

TABLE 5
Frequency of wild type (intact) and polymorphic(disrupted) olfactory
receptor (OR) alleles in African Americans and Non-Africans

| | | | | | Intact Allele | | | Disrupted Allele | | |
|---|---|---|---|---|---|---|---|---|---|---|
| | chromosomal | | SNP | | African- | Non- | | African- | Non- | |
| Gene name | location | SNP (INT > pse) | position | SNP type | Americans | African | Total | Americans | African | Total |
| OR8J2p | 11 + 61.7 | C > T | 208 | STOP | 0.1 | 0.04 | 0.07 | 0.9 | 0.96 | 0.93 |
| OR4A8p | 11 + 58.9 | C > T | 406 | STOP | 0.13 | 0.12 | 0.12 | 0.87 | 0.88 | 0.88 |
| OR1E3p | 17 + 3.3 | C > DEL | 54 | DEL | 0.28 | 0.09 | 0.19 | 0.72 | 0.91 | 0.81 |
| OR1P1p | 17 + 3.3 | A > T | 553 | STOP | 0.29 | 0.18 | 0.24 | 0.71 | 0.82 | 0.76 |
| OR5H6 | 3 + 104 | T > C | 535 | C > R | 0.32 | 0.34 | 0.33 | 0.68 | 0.66 | 0.67 |
| OR12D2 | 6 + 32.9 | G > T | 359 | (DRY) R > L | 0.29 | 0.42 | 0.35 | 0.71 | 0.58 | 0.65 |
| OR11H7p | 14 + 17.6 | C > T | 691 | STOP | 0.42 | 0.46 | 0.44 | 0.58 | 0.54 | 0.56 |
| OR10X1p | 1 + 154 | G > A | 147 | STOP | 0.42 | 0.48 | 0.45 | 0.58 | 0.52 | 0.55 |
| OR1S1 | 11 + 64.1 | G > A | 404 | (DRY) R > H | 0.36 | 0.54 | 0.45 | 0.64 | 0.46 | 0.55 |
| OR3A1 | 17 + 3.3 | G > A | 374 | (DRY) R > Q | 0.68 | 0.4 | 0.55 | 0.32 | 0.6 | 0.45 |
| OR4Q2p | 14 + 17.6 | DEL > T | 515 | INS | 0.65 | 0.47 | 0.56 | 0.35 | 0.53 | 0.44 |
| OR4K3 | 14 + 17.6 | C > DEL | 622 | DEL | 0.51 | 0.67 | 0.58 | 0.49 | 0.33 | 0.42 |
| OR5G3 | 11 + 61.7 | C > DEL | 373 | DEL | 0.68 | 0.64 | 0.66 | 0.32 | 0.36 | 0.34 |
| OR51F1p | 11 + 15.1 | C > DEL | 274 | DEL | 0.59 | 0.83 | 0.7 | 0.41 | 0.17 | 0.3 |
| OR2J1p | 6 + 32.9 | C > T | 574 | STOP | 0.82 | 0.59 | 0.71 | 0.18 | 0.41 | 0.29 |
| OR52B4 | 11 + 15.1 | C > DEL | 119 | DEL | 0.82 | 0.63 | 0.73 | 0.18 | 0.37 | 0.27 |
| OR1B1 | 9 + 106 | C > T | 574 | STOP | 0.89 | 0.59 | 0.74 | 0.11 | 0.41 | 0.26 |
| OR8K3 | 11 + 61.7 | G > T | 365 | (DRY) R > L | 0.78 | 0.72 | 0.75 | 0.22 | 0.28 | 0.25 |
| OR52N4 | 11 + 15.1 | A > T | 514 | STOP | 0.82 | 0.72 | 0.77 | 0.18 | 0.28 | 0.23 |
| OR52H1 | 11 + 15.1 | G > A | 389 | (DRY)R > H | 0.92 | 0.88 | 0.9 | 0.08 | 0.12 | 0.1 |
| OR6Q1 | 11 + 64.1 | C > DEL | 685 | DEL | 0.9 | 0.94 | 0.92 | 0.1 | 0.06 | 0.08 |
| OR7C2 | 19 + 17 | G > A | 365 | (DRY) R > H | 0.87 | 0.98 | 0.92 | 0.13 | 0.02 | 0.08 |
| OR51G1 | 11 + 15.1 | G > A | 371 | (DRY) R > H | 0.96 | 0.92 | 0.94 | 0.04 | 0.08 | 0.06 |
| OR5AL1p | 11 + 61.7 | CT > DEL | 421 | DEL | 0.96 | 0.91 | 0.94 | 0.04 | 0.09 | 0.06 |
| OR10C1 | 6 + 32.9 | C > T | 163 | STOP | 0.93 | 0.98 | 0.95 | 0.07 | 0.02 | 0.05 |
| OR2A9p | 7 + 149 | C > T | 277 | STOP | 0.94 | 0.96 | 0.95 | 0.06 | 0.04 | 0.05 |

TABLE 6

Oligonucleotide sequences useful in identifying both intact and
pseudogene alleles of the OR genes of Table 5

| | | | |
|---|---|---|---|
| | | | Oligonucleotide for |
| Gene Name | Intact oligo | Pseudogene oligo | primer extension assay |

| | | | | | | |
|---|---|---|---|---|---|---|
| OR10C1 | GATGCTGCCCTCCAGTCCCCTATGT | (1) | GATGCTGCCCTCTAGTCCCCTATGT | (27) | TCCACTGATGCTGCCCTC | (53) |
| (79) | | | | | | |
| OR10X1p | GGGTCTAACTTGGGTGGACAGGTCC | (2) | GGGTCTAACTTGAGTGGACAGGTCC | (28) | CCATCATGGGTCTAACTTG | (54) |
| (80) | | | | | | |
| OR11H7p | AGAACTGTGCTTCAGGTTCCTTCTT | (3) | AGAACTGTGCTTTAGGTTCCTTCTT | (29) | CAGCTGAAGAAGGAACCT | (55) |
| (81) | | | | | | |
| OR12D2 | GGCATTTGACCGCTCTGTGGCTAT | (4) | GGCATTTGACCTCTCTGTGGCTAT | (30) | GCCGTGATGGCATTTGACC | (56) |
| (82) | | | | | | |
| OR1B1 | CGGCCACTTCTGCGAGCCTCTTGTT | (5) | CGGCCACTTCTGTGAGCCTCTTGTT | (31) | TGTCAGAACAAGAGGCTC | (57) |
| (83) | | | | | | |
| OR1E3p | CCTGGGCCTTCCCATCCAACCTGAG | (6) | CCTGGGCCTTCCATCCAACCTGAG | (32) | CCTGCTCCTGGGCCTTCC | (58) |
| (84) | | | | | | |
| OR1P1p | TGCTCTAAGATCCTTCCTGGCTTCTTC | (7) | TGCTCTTAGATCCTTCCTGGCTTCTTC | (33 | AGAAGCCAGGAAGGATCT | (59) |
| (85) | | | | | | |
| OR1S1 | TGGCCTATGACCGCTTTGTGGCGAT | (8) | TGGCCTATGACCACTTTGTGGCGAT | (34) | TGGCAGATCGCCACAAAG | (60) |
| (86) | | | | | | |
| OR2A9p | CTGCATGACACAGACCTTTCTCTTT | (9) | CTGCATGACATAGACCTTTCTCTTT | (35) | ACTCAAAAAGAGAAAGGTCT | (61) |
| (87) | | | | | | |
| OR2J1pI | TGTGTTGATACCCAGGCAAATGAGC | (10) | TGTGTTGATACCTAGGCAAATGAGC | (36) | GATTATCATGTGTTGATACC | (62) |
| (88) | | | | | | |
| OR3A1 | TGGCCTATGACCGATTCCTGGCCAT | (11) | TGGCCTATGACCAATTCCTGGCCAT | (37) | CGGCAGATGGCCAGGAAT | (63) |
| (89) | | | | | | |
| OR4A8p | ACCATCATGAATCGACAGGTTTGCA | (12) | ACCATCATGAATTGACAGGTTTGCA | (38) | GAAGGATGCAAACCTGTC | (64) |
| (90) | | | | | | |
| OR4K3 | GGCATGATTGCCCTGGTGTGCTTCA | (13) | GGCATGATTGCCTGGTGTGCTTCA | (39) | AGAGGATGAAGCACACCA | (65) |
| (91) | | | | | | |
| OR4Q2p | AAAACTTTTTTTGTGATGTTCCCC | (14) | AAAACTTTTTTTTGTGATGTTCCCC | (40) | ATCAGGTGGAAAACTTTTTTT | (66) |
| (92) | | | | | | |
| OR51F1p | TGGTTTGAGGCACGTGAAATCAGTCT | (15) | TGGTTTGAGGCAGTGAAATCAGTCT | (41) | ATCCTCTGGTTTGAGGCA | (67) |
| (93) | | | | | | |
| OR51G1 | TGTCCATTGACCGCTACGTGGCCGT | (16) | TGTCCATTGACCACTACGTGGCCGT | (42) | TTATCCATGTCCATTGACC | (68) |
| (94) | | | | | | |
| OR52B4 | CCTATGTCACCGCCCTTCTTGGGAA | (17) | CCTATGTCACCGCTTCTTGGGAA | (43) | AGGCTGTTCCCAAGAAGG | (69) |
| (95) | | | | | | |
| OR52H1 | TGGCATTTGATCGCTATGTAGCTAT | (18) | TGGCATTTGATCACTATGTAGCTAT | (44) | TGGCCATGGCATTTGATC | (70) |
| (96) | | | | | | |
| OR52N4 | CTGCCCTACTGCAGAGGCAATATAC | (19) | CTGCCCTACTGCTGAGGCAATATAC | (45) | TGGGGAAGTATATTGCCTC | (71) |
| (97) | | | | | | |
| OR5AL1p | ATTCCTAGAAAACTCTGTATTAAA | (20) | ATTCCTAGAAAACTGTATTAAA | (46) | GTCATTCTCATTCCTAGAAAA | (72) |
| (98) | | | | | | |
| OR5G3 | GAGTGTTTTCTCCTGGCTGCCATGG | (21) | GAGTGTTTTCTCTGGCTGCCATGG | (47) | GCAGCTGAGTGTTTTCTC | (73) |
| (99) | | | | | | |
| OR5H6 | CAACACTTTTACTGTGACATTATCC | (22) | CAACACTTTTACCGTGACATTATCC | (48) | TAACAATGGGATAATGTCAC | (74) |
| (100) | | | | | | |
| OR6Q1 | ATCGTCTGGACACTGCTGCACATCC | (23) | ATCGTCTGGACATGCTGCACATCC | (49) | GGCAACATCGTCTGGACA | (75) |
| (101) | | | | | | |
| OR7C2 | CGGCCTATGACCGCTTCGTGGCCAT | (24) | CGGCCTATGACCACTTCGTGGCCAT | (50) | TAACAGATGGCCACGAAG | (76) |
| (102) | | | | | | |
| OR8J2p | TACTTTTTCCTCCGACACTTGGCTA | (25) | TACTTTTTCCTCTGACACTTGGCTA | (51) | CCCCATGTACTTTTTCCTC | (77) |
| (103) | | | | | | |
| OR8K3 | TGTCCTACGACCGCTATGTGGCCAT | (26) | TGTCCTACGACCTCTATGTGGCCAT | (52) | GTTACAGATGGCCACATAG | (78) |
| (104) | | | | | | |

SEQ ID NO of ORs and oligonucleotide sequences indicated within parentheses

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

### Additional References Cited Herein

1. Abecasis, G. R. and Cookson, W. O. (2000). GOLD—graphical overview of linkage disequilibrium. Bioinformatics, 16: 182-183.
2. Alewijnse, A. E., Timmerman, H. et al., (2000). The effect of mutations in the DRY motif on the constitutive activity and structural instability of the histamine H(2) receptor. Mol. Pharmacol. 57: 890-898.
   3. Amoore J E (1977) Specific anosmia and the concept of primary odors. Chem. Senses Flavor 2:267-281
4. Braverman J M, Hudson R R, Kaplan N L, Langley C H, Stephan W (1995) The hitchhiking effect on the site frequency spectrum of DNA polymorphisms. Genetics 140:783-796
5. Buck L, Axel R (1991) A novel multigene family may encode odorant receptors: a molecular basis for odor recognition. Cell 65:175-87
6. Clark, A. G., Weiss, K. M., Nickerson, D. A., et al., (1998) Haplotype structure and population genetic inference from nucleotide sequence variation in human lipoprotein lipase. Am. J. Hum. Genet. 63: 595-612.
7. Clark, A. G. ( 1990). Inference of haplotypes from PCR-amplified samples of dipoid populations. Mol. Biol. Evol. 7: 111-122.
8. Ewens, W. J. ( 1982). On the concept of the effective population size. Theor. Population Biol. 21: 373-378.
9. Excoffier, L. a. M. S. (1995). Maximum-likelihood estimation of molecular haplotype frequencies in diploid population. Mol. Biol. Evol. 12: 921-927.
10. Fay J C, Wu C I (2000) Hitchhiking under positive Darwinian selection. Genetics 155:1405-13
11. Firestein S (2001) How the olfactory system makes sense of scents. Nature 413:211-8.
12. Frisse L, Hudson R R, Bartoszewicz A, Wall J D, Donfack J, Di Rienzo A (2001) Gene conversion and different population histories may explain the contrast between polymorphism and linkage disequilibrium levels. Am J Hum Genet 69:831-43.
13. Fullerton, S. M., Clark, A. G., Weiss, K. M., et al., (2000). Apolipoprotein E variation at the sequence haplotype levele: implications for the origin and maintenance of a major human polymorphism. Am. J. Hum. Genet. 67: 881-900.
14. Gilad, Y. and Lancet, D. (2003). Population differences in the human functional olfactory repertoire. Mol. Biol. Evol. 20: 307-314.
15. Gilad Y, Rosenberg S, Przeworski M, Lancet D, Skorecki K (2002) Evidence for positive selection and population structure at the human MAO-A gene. Proc Natl Acad Sci USA 99:862-867.
16. Gilad Y, Segre D, Skorecki K, Nachman M W, Lancet D, Sharon D (2000) Dichotomy of single-nucleotide polymorphism haplotypes in olfactory receptor genes and pseudogenes. Nat Genet 26:221-4.
17. Glusman G, Bahar A, Sharon D, Pilpel Y, White J, Lancet D (2000) The olfactory receptor gene superfamily: data mining, classification, and nomenclature. Mamm Genome 11: 1016-23.
18. Glusman G, Yanai I, Rubin I, Lancet D (2001) The complete human olfactory subgenome. Genome Res 11:685-702.
19. Gross-Isseroff R, Ophir, D., Bartana, A., Voet, H. and Lancet, D (1992) Evidence for Genetic Determination in Human Twins of Olfactory Thresholds for a Standard Odorant. Neurosci. Lett. 141:115-118
20. Hamblin M T, Thompson E E, Di Rienzo A (2002) Complex signatures of natural selection at the Duffy blood group locus. Am J Hum Genet 70:369-83.
21. Hammer, M. F., Redd, A. J., Wood, E. T. et al., (2000). Jewish and Middle Eastern non-Jewish populations share a common pool of Y-chromosome biallelic haplotypes. Proc. Natl. Acad. Sci. USA. 97: 6769-6774.
22. Harding R M, Fullerton S M, Griffiths R C, Bond J, Cox M J, Schneider J A, Moulin D S, Clegg J B (1997) Archaic African and Asian lineages in the genetic ancestry of modem humans. Am. J. Hum. Genet. 60:772-789
23. Harding R M, Healy E, Ray A J, Ellis N S, Flanagan N, Todd C, Dixon C, Sajantila A, Jackson U, Birch-Machin M A, Rees J L (2000) Evidence for variable selective pressures at MC1R. Am J Hum Genet 66:1351-61
24. Hudson, R. R. and Kaplan, L. L. (1985). Statistical properties of the number of recombination events in the history of a sample of DNA sequence. Genetics, 111:147-164.
25. Hudson, R. R. (2000). A new statistic for detecting genetic differentiation. Genetics, 155: 2011-2014.
26. Kobyliansky, E., Micle, S et al., (1982). Jewish populations of the world: genetc likeness and differences. Ann. Hum. Biol. 9: 1-34.
27. Lancet D, Ben-Arie N (1993) Olfactory receptors. Current Biology 3:668-674
28. Lancet D, Ben-Arie N, Cohen S, Gat U, Gross-Isseroff R, Horn-Saban S, Khen M, Lehrach H, Natochin M, North M, et al. (1993a) Olfactory receptors: transduction, diversity, human psychophysics and genome analysis. Ciba Found Symp 179:131-41
29. Lancet D, Gross-Isseroff R, Margalit T, Seidmann E, Ben-Arie N (1993b) Olfaction: from signal transduction and termination to human genome mapping. Chem Senses 18:217-225
30. Lander E S, Linton L M, Birren B, Nusbaum C, Zody M C, Baldwin J, Devon K, et al. (2001) Initial sequencing and analysis of the human genome. Nature 409:860-921.
31. Lewontin, R. C. (1964). The interaction of selection and linkage. I. General considerations: heterotic models. Genetics 49: 49-67.
32. Maynard-Smith J M, Haigh J (1974) The hitch-hiking effect of a favourable gene. Genet Res 23:23-35
33. Menashe I, Oma M, Lancet D, Gilad Y (2002) Population differences in haplotype structure within a human olfactory receptor gene cluser. Hum. Mol. Genet. In Press
34. Mombarts P (1999) Seven-transmembrane proteins as odorant and chemosensory receptors. Science 286:707-711
35. Nachman M W, Gilad Y, Rosenberg S, Przeworski M, Lancet D, Skorecki K (2001) Single nucleotide polymorphisms and recombination rate in humans. Trends Genet,17:481-5. positive selection.
36. Nei M, Li W H (1979) Mathematical model for studying genetic variation in terms of restriction endonucleases. Proc. Natl. Acad. Sci. 76:5269-5273
37. Nickerson D A, Taylor S L, Fullerton S M, Weiss K M, Clark A G, Stengard J H, Salomaa V, Boerwinkle E, Sing C F (2000) Sequence diversity and large-scale typing of SNPs in the human apolipoprotein E gene [In Process Citation]. Genome Res 10:1532-45
38. Nickerson D A, Taylor S L, Weiss K M, Clark A G, Hutchinson R G, Stengard J, Salomaa V, Vartiainen E, Boerwinkle E, Sing C F (1998) DNA sequence diversity in a 9.7-kb region of the human lipoprotein lipase gene. Nature Genet. 19:233-40
39. Orr H A, Betancourt A J (2001) Haldane's sieve and adaptation from the standing genetic variation. Genetics 157:875-84.
40. Payseur B A, Nachman M W (2000) Microsatellite variation and recombination rate in the human genome. Genetics in press
41. Pilpel Y, Lancet D (1999) The variable and conserved interfaces of modeled olfactory receptor proteins. Protein Science 8:969-977
42. Pritchard J K, Przeworski M (2001) Linkage disequilibrium in humans: models and data. Am J Hum Genet 68:1-14
43. Probst, W. C. Snyder, L. A. et al., (1992). Sequence alignment of the G-protein coupled receptor superfamily. DNA Cell Biol. 11: 1-20.
44. Przeworski M (2002) The signature of positive selection at randomly chosen loci. Genetics 160:1179-1189
45. Przeworski M, Hudson R R, Di Rienzo A (2000) Adjusting the focus on human variation. Trends Genet 16:296-302.
46. Reich, D. E., Cargill, M. et al., (2001). Linkage disequilibrium in the human genome. Nature 411: 199-204.
47. Reich D E, Goldstein D B (1998) Genetic evidence for a Paleolithic human population expansion in Africa. Proc Natl Acad Sci USA 95:8119-23.
48. Rieder, M. J., Taylor, S. L. et al., (1999). Sequence variation in the human angiotensin converting enzyme. Nat. genet. 22: 59-62.
49. Risch N, Merikangas K, Tishkoff S A, Varkonyi R, Cahinhinan N, Abbes S, Argyropoulos G, Destro-Bisol G, Drousiotou A, Dangerfield B, Lefranc G, Loiselet J, Piro A, Stoneking M, Tagarelli A, Tagarelli G, Touma E H, Williams S M, Clark A G (I996) The future of genetic studies of complex human diseases. Science 273:1516-7.
50. Rouquier S, Blancher A, Giorgi D (2000) The olfactory receptor gene repertoire in primates and mouse: evidence for reduction of the functional fraction in primates. Proc Natl Acad Sci USA 97:2870-4.
51. Rozas, J. and Rozas, R. (1995). DnaSP, DNA sequence polymorphism: an interactive program for estimating population genetics parameters from DNA sequence data. Comput. Appl. Biosci. 11: 621-625.
52. Sbrahmanyan, L., Eberle, M. A., Clark, A. G. et al., (2001). Sequence variation and linkage disequilibrium in the human T-cell receptor beta (TCRB) locus. AM. J. Hum. Genet. 69: 381-395.
53. Schoneberg, T., Schulz, A., Gudermann, T. (2002). The structural basis of G-protein-coupled receptor function and dysfunction in human diseases. Rev. Physiol. Biochem. Pharmacol. 144: 143-227.
54. Sharon D, Glusman G, Pilpel Y, Khen M, Gruetzner F, Haaf T, Lancet D (1999) Primate Evolution of an Olfactory Receptor Cluster: Diversification by Gene Conversion and Recent Emergence of Pseudogenes. Genomics 61:24-36
55. Slatkin M, Hudson R R (1991) Pairwise comparisons of mitochondrial DNA sequences in stable and exponentially growing populations. Genetics 129:555-62.
56. Stephens J C, Schneider J A, Tanguay D A, Choi J, Acharya T, Stanley S E, Jiang R, et al. (2001) Haplotype variation and linkage disequilibrium in 313 human genes. Science 293:489-93.
57. Tajima F (1989a) The effect of change in population size on DNA polymorphism. Genetics 123:597-601
58. Tajima F (1989b) Statistical method for testing the neutral mutation hypothesis by DNA polymorphism. Genetics 123:585-595.
59. Tajima, F. (1993). Measurment of DNA polymorphism. In Takahata, N. and Clark, A. G. (eds). Mechanisms of Molecular Evolution. Japan Scientific Sicieties Press. Tokyo, pp. 37-60.
60. Tishkoff S A, Varkonyi R, Cahinhinan N, Abbes S, Argyropoulos G, Destro-Bisol G, Drousiotou A, Dangerfield B, Lefranc G, Loiselet J, Piro A, Stoneking M, Tagarelli A, Tagarelli G, Touma E H, Williams S M, Clark A G (2001) Haplotype diversity and linkage disequilibrium at human G6PD: recent origin of alleles that confer malarial resistance. Science 293:455-62.
61. Wall J D (1999) Recombination and the power of statistical tests of neutrality. Genet Res 74:65-79
62. Wall J D, Przeworski M (2000) When did the human population size start increasing? Genetics 155:1865-74.
63. Watterson GA (1975) On the number of segregating sites in genetical models without recombination. Theoretical population biology 7:256-276
64. Whissell-Buechy D, Amoore J E (1973) Letter: Odour-blindness to musk: simple recessive inheritance. Nature 245:157-8
65. Wright, S. (1951). The genetical structure of populations. Ann. Eugenics 15: 323-354.
66. Wysocki C J, Beauchamp G K (1984) Ability to smell androstenone is genetically determined. Proc Natl Acad Sci USA 81:4899-902
67. Venter, J. C. et al., (2001). Science 291: 1304-51.
68. Yeager, M. and Hughes, A. L. (1999). Evolution of the mammalian MHC: natural section, recombination, and convergent evolution. Immunol. Rev. 167: 45-58.
69. Young J M, Friedman C, Williams E M, Ross J A, Tonnes-Priddy L, Trask B J (2002) Different evolutionary processes shaped the mouse and human olfactory receptor gene families. Hum Mol Genet 11:535-46
70. Younger R M, Amadou C, Bethel G, Ehlers A, Lindahl K F, Forbes S, Horton R, Milne S, Mungall A J, Trowsdale J, Volz A, Ziegler A, Beck S (2001) Characterization of clustered MHC-linked olfactory receptor genes in human and mouse. Genome Res 11:519-30.
71. Zhang X, Firestein S (2002) The olfactory receptor gene superfamily of the mouse. Nat Neurosci 5:124-33.
72. Zozulya S, Echeverri F, Nguyen T (2001) The human olfactory receptor repertoire. Genome Biol 2:RESEARCH0018

1. An oligonucleotide comprising a nucleic acid sequence selected suitable for identifying an olfactory receptor gene or an allelic variant thereof, said olfactory receptor gene being selected from the group consisting of SEQ ID NOs: 79-104.

2. The oligonucleotide of claim 1, further comprising a detectable moiety attached to said nucleic acid sequence.

3. The oligonucleotide of claim 2, wherein said detectable moiety is selected from the group consisting of a dye, a fluorophore, an enzyme, a ligand and a radioisotope.

4. The oligonucleotide of claim 1, wherein the oligonucleotide is selected from the group consisting of SEQ ID NOs: 1-78.

5. The oligonucleotide of claim 1, wherein the oligonucleotide is an SNP-specific oligonucleotide.

6. The oligonucleotide of claim 1, wherein the oligonucleotide is a primer extension oligonucleotide.

7. A kit for identifying an olfactory receptor gene and/or an allelic variant thereof, the kit comprising at least oligonucleotide having a nucleic acid sequence selected suitable for identifying the olfactory receptor gene and/or the allelic variant thereof.

8. The kit of claim 7, further comprising reagents suitable for detecting identification of the olfactory receptor gene and/or the allelic variant thereof by said at least one oligonucleotide.

9. The kit of claim 7, further comprising packaging material identifying said at least oligonucleotide as being utilizable in detecting the olfactory receptor gene and/or the allelic variant thereof.

10. The kit of claim 7, wherein the olfactory receptor gene is selected from the group consisting of SEQ ID NOs: 79-104.

11. The kit of claim 7, wherein said at least one oligonucleotide is selected from the group consisting of SEQ ID NOs: 1-78.

12. The kit of claim 7, wherein said at least oligonucleotide includes a detectable moiety attached to said nucleic acid sequence.

13. The kit of claim 12, wherein said detectable moiety is selected from the group consisting of a dye, a fluorophore, an enzyme, a ligand and a radioisotope.

14. An array for detecting the presence or absence of at least one allelic variant of an olfactory receptor gene in a subject, the array comprising at least one oligonucleotide being contained in or attached to a support, said at least oligonucleotide having a nucleic acid sequence selected suitable for specifically identifying the at least one allelic variant of the olfactory receptor gene.

15. The array of claim 14, further comprising at least one additional oligonucleotide having a nucleic acid sequence selected suitable for specifically identifying the olfactory receptor gene.

16. The array of claim 15, wherein the olfactory receptor gene is selected from the group consisting of SEQ ID NOs: 79-104.

17. The array of claim 14, wherein said at least one oligonucleotide is selected from the group consisting of SEQ ID NOs: 27-78.

18. An array for typing a subject according to presence or absence of allelic variants of olfactory receptor genes, the array comprising a plurality of oligonucleotides each being attached to a support, said plurality of oligonucleotides include at least one typing oligonucleotide having a sequence selected suitable for specifically identifying presence or absence of a specific allelic variant of a specific olfactory receptor gene in the subject.

19. The array of claim 18, wherein said plurality of oligonucleotides also include at least one reference oligonucleotide having a sequence selected suitable for specifically identifying said specific olfactory receptor gene.

20. The array of claim 18, wherein said support is a chip.

21. The array of claim 18, wherein said at least one typing oligonucleotide is selected from the group consisting of SEQ ID NOs: 27-78.

22. The array of claim 19, wherein said at least one reference oligonucleotide is selected from the group consisting of SEQ ID NOs: 1-26.

23. A method of typing a subject according to presence or absence of allelic variants of olfactory receptor genes, the method comprising detecting the presence or absence of at least one allelic variant of an olfactor receptor gene in a biological sample of the subject thereby typing the subject.

24. The method of claim 23, further comprising detecting the presence or absence of said olfactory receptor gene in said biological sample of the subject.

25. The method of claim 23, wherein the olfactory receptor gene is selected from the group consisting of SEQ ID NOs: 79-104.

26. The method of claim 23, wherein said detecting the presence or absence of at least one allelic variant of said olfactory receptor gene is effected using at least one oligonucleotide selected from the group consisting of SEQ ID NOs: 27-78.

27. The method of claim 24, wherein said detecting the presence or absence of said olfactory receptor gene is effected using at least one oligonucleotide selected from the group consisting of SEQ ID NOs: 1-26.

28. The method of claim 23, wherein said detecting the presence or absence of at least one allelic variant of said olfactory receptor gene is effected by detecting DNA and/or mRNA sequences.

29. The method of claim 23, wherein said detecting the presence or absence of at least one allelic variant is effected using at least one oligonucleotide selected from the group consisting of SEQ ID NOs: 27-78.

30. The method of claim 24, wherein said detecting the presence or absence of said olfactory receptor gene is effected using at least one oligonucleotide selected from the group consisting of SEQ ID NOs: 1-26.

31. A nucleic acid construct comprising a polynucleotide encoding an olfactory receptor gene and/or an allelic variant thereof, said olfactory receptor gene being selected from the group consisting of SEQ ID Nos: 79-104 and a promoter for directing transcription of said olfactory receptor gene or said allelic variant thereof in a cell.

32. A cell comprising the nucleic acid construct of claim 31.

33. The cell of claim 31, wherein the cell is a mammalian cell.
